(19) United States
(12) Patent Application Publication
(10) Pub. No.: US 2010/0105651 A1
(43) Pub. Date: Apr. 29, 2010

(54) ANTAGONISTS OF SNS SODIUM CHANNELS

(75) Inventors: Richard Hamlyn, Cambridge (GB); David Callis, Cambridge (GB); Christopher Geoffrey Earnshaw, Cambridge (GB); Harry Finch, Cambridge (GB); Mike Huckstep, Cambridge (GB); Rosemary Lynch, Cambridge (GB); Sarah Mellor, Cambridge (GB)

Correspondence Address: BANNER & WITCOFF, LTD., 1100 13th STREET, N.W., SUITE 1200, WASHINGTON, DC, 20005-4051, US

(73) Assignee: VERNALIS (R&D) LIMITED, Winnersh (GB)

(21) Appl. No.: 11/993,515

(22) PCT Filed: Jul. 7, 2006
(86) PCT No.: PCT/GB2006/002523

§ 371 Date: Dec. 22, 2009

(30) Foreign Application Priority Data
Jul. 7, 2005 (GB) 0514017.3

(51) Int. Cl. A61K 31/397; C07D 205/04; C07D 401/02; A61P 1/00; A61P 13/10; A61P 25/00

(52) U.S. Cl. 514/210.18; 548/953; 546/139; 546/208; 514/210.17

(57) ABSTRACT

Compounds of the formula (I), and pharmaceutically acceptable salts thereof, are found to be antagonists of SNS sodium channels. They are therefore useful as analgesic and neuroprotective agents, formula (I): R¹ represents: (a) -L-A or -L′-A′ wherein L represents a bond or a C₁-C₆ alkyl, C₂-C₆ alkenyl or C₂-C₆ alkynyl moiety, A represents a phenyl, 5- to 10-membered heteroaryl, C₃-C₆ carbocyclyl or 5- to 10-membered heterocyclyl group, L′ represents a C₁-C₆ alkyl, C₂-C₆ alkenyl or C₂-C₆ alkynyl moiety, and A′ represents -Het-A or —X-A wherein Het represents —O—, —S— or —NR¹—, and X represents —CO—, —SO—, —SO₂—, —CO—O—, —CO—S—, —CONR¹—, —O—CO—, —S—CO— or —NR¹—CO—, wherein R¹ represents hydrogen or C₁-C₆ alkyl; (b) -L-CR(A)(A′) or -L-CR(A)(L-A) wherein R is hydrogen or C₁-C₄ alkyl, A′ is as defined above, each L is the same or different and is as defined above and each A′ is the same or different and is as defined above; (c) -L-A-A′ or -L-A-L-A wherein A′ and L are as defined above and each A is the same or different and is as defined above; or (d) -A-Z-A wherein Z is -Het-L′-, —X-L′-, -L′-Het- or -L′-X—, wherein Het, L′ and X are as defined above and each A is the same or different and is as defined above; J represents —NR^{S}—, —O— or a direct bond; R⁵ represents hydrogen, C₁-C₆ alkyl, C₂-C₆ alkenyl or C₂-C₆ alkynyl; R⁴ is represents hydrogen, C₁-C₆ alkyl, C₂-C₆ alkenyl or C₂-C₆ alkynyl; and either R represents -L-A, -L-A′, -L-A-A′, -L-A-L-A, -L-CR(A)(L-A), -L-CR(A)(A′) or -L-CR(A)(L″) wherein L″ is -Het-L′, —CONH₂ or —CO₂H, and wherein A′, Het, X and R are as defined above, each L is the same or different and is as defined above, each A is the same or different and is as defined above and R³ represents hydrogen, C₁-C₆ alkyl C₂-C₆ alkynyl or (CO)-L′, wherein L is as defined above or R² and R³ form, together with the nitrogen to which they are attached, a 5- to 10-membered heteroaryl or 5- to 10-membered heterocyclyl ring.

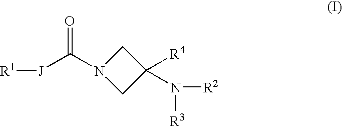

The present invention relates to inhibitors of the subtype of mammalian sodium channels known as Naᵥ1.8 or sensory neurone specific (SNS) channels. The Naᵥ1.8 channel is a 1,957 amino acid tetrodotoxin-insensitive voltage-gated sodium channel. The sodium channel, nucleic acid sequences coding for the channel, vectors, host cells and methods of identifying modulators, are taught in U.S. Pat. No. 6,451,554. The α-subunit gene corresponding to this ion channel is referred to as SCN10A. The channel is described in more detail in Akopian et al., (1996), 379, 257-262.

Mammalian ion channels are becoming increasingly well characterized, and progress in sodium channel research has been summarized recently in Anger et al, J. Med. Chem. (2001) 44, 115-137. Sodium channels are recognised as valid targets for pain therapeutics, and blockade of sodium channels can be useful in the treatment of a range of pain syndromes (see for example Black et al, Progress in Pain Research and Management (2001), 21 (Neuropathic Pain: Pathophysiology and Treatment), 19-36).

It has now surprisingly been found that compounds of the general formula (I) set out below act as inhibitors of sensory neurone specific sodium channels. Accordingly, the present invention provides the use, in the manufacture of a medicament for use in the treatment or prevention of a condition involving sodium ion flux through a sensory neurone specific channel of a sensory neurone, of a compound of the formula (I), or a pharmaceutically acceptable salt thereof

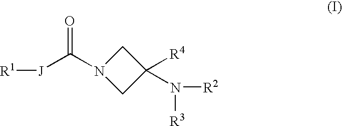

R¹ represents:

(a) -L-A or -LP-A′ wherein L represents a bond or a C₁-C₆ alkyl, C₂-C₆ alkenyl or C₂-C₆ alkynyl moiety, A represents a phenyl, 5- to 10-membered heteroaryl, C₃-C₆ carbocyclyl or 5- to 10-membered heterocyclyl group, L′ represents a C₁-C₆ alkyl, C₂-C₆ alkenyl or C₂-C₆ alkynyl moiety, and N represents -Het-A or —X-A wherein Het represents —O—, —S— or —NR¹—, and X represents —CO—, —SO—, —SO₂—, —CO—O—, —CO—S—, —CONR¹—, —O—CO—, —S—CO— or —NR¹—CO—, wherein R¹ represents hydrogen or C₁-C₆ alkyl;

(b) -L-CR(A)(A′) or -L-CR(A)(L-A) wherein R is hydrogen or C₁-C₄ alkyl, A′ is as defined above, each L is the same or different and is as defined above and each A is the same or different and is as defined above;

(c) -L-A-A′ or -L-A-L-A wherein A′ and L are as defined above and each A is the same or different and is as defined above; or

(d) -A-Z-A wherein Z is -Het-L′-, —X-L′-, -L′-Het- or -L′-X—, wherein Het, L′ and X are as defined above and each A is the same or different and is as defined above;
J represents —NR⁵—, —O— or a direct bond wherein R⁵ represents hydrogen, C₁-C₆ alkyl, C₂-C₆ alkenyl or C₂-C₆ alkynyl;
R⁴ is represents hydrogen, C₁-C₆ alkyl, C₂-C₆ alkenyl or C₂-C₆ alkynyl; and either —R² represents -L-A, -L-A′, -L-A-A′, -L-A-L-A, -L-CR(A)(L-A), -L-CR(A)(A′) or -L-CR(A)(L″) wherein L″ is -Het-L′, —X-L′, —CONH₂ or —CO₂H, and wherein A′, Het, X and R are as defined above, each L is the same or different and is as defined above, each A is the same or different and is as defined above and each L′ is the same or different and is as defined above, and

R³ represents hydrogen, C₁-C₆ alkyl, C₂-C₆ alkenyl, C₂-C₆ alkynyl or —(CO)-L′, wherein L′ is as defined above, or

R² and R³ form, together with the nitrogen to which they are attached, a 5- to 10-membered heteroaryl or 5- to 10-membered heterocyclyl ring,

wherein:

said phenyl, carbocyclyl, heterocyclyl and heteroaryl groups are optionally fused to a further cyclic moiety selected from phenyl, C₅-C₆ carbocyclyl, 5- to 6-membered heterocyclyl and 5- to 6-membered heteroaryl groups; the phenyl, heteroaryl, carbocyclyl and heterocyclyl groups and moieties in the groups R¹, R² and that formed by R² and R³ are unsubstituted or substituted by one, two or three substituents which are the same or different and are selected from halogen, hydroxy, amino, thio, C₁-C₆ alkyl, C₂-C₆ alkenyl, nitro, cyano or -Het-L′, wherein Het and L′ are as defined above; and

the alkyl, alkenyl and alkynyl groups and moieties in R¹ to R⁵ are unsubstituted or substituted by one, two or three substituents which are the same or different and are selected from halogen, hydroxy, amino and thio substituents.

For the avoidance of doubt, when A′ represents —X-A, the orientation of the group X is such that the right hand side of the depicted moiety is attached to A. Thus, for example, when X is —CO—O—, the group —X-A is —CO—O-A.

For the avoidance of doubt, when R¹ represents -A-Z-A, the orientation of the group Z is such that the left hand side of the depicted group is attached to the divalent A group. Thus, for example, when Z is -Het-L′-, the group -A-Z-A is -A-Het-L′-A.

For the avoidance of doubt, when Z represents —X-L′-, the orientation of the group X is such that the right hand side of the depicted moiety is attached to L′. Thus, for example, when X is —CO—O—, the group —X-L′ is —CO—O-L.

For the avoidance of doubt, when Z represents -L′-X—, the orientation of the group X is such that the left hand side of the depicted moiety is attached to L′. Thus, for example, when X is —CO—O—, the group -L′-X— is -L′-CO—O—.

For the avoidance of doubt, when L″ represents —X-L′, the orientation of the group X is such that the right hand side of the depicted moiety is attached to L′. Thus, for example, when X is —CO—O—, the group —X-L′ is —CO—O-L′.

As used herein, a C₁-C₆ alkyl group or moiety is a linear or branched alkyl group or moiety containing from 1 to 6 carbon atoms, such as C₁-C₄ alkyl group or moiety, for example methyl, ethyl, n-propyl, i-propyl, n-butyl, i-butyl and t-butyl. Preferred C₁-C₆ alkyl groups are methyl, ethyl, n-propyl and n-butyl. A divalent alkyl moiety (or alkylene moiety) can be attached via the same carbon atom, by adjacent carbon atoms or by non-adjacent carbon atoms. Preferred divalent alkyl groups are methylene, 1,1-ethylene and 2,2-propylene groups.

As used herein, a C₂-C₆ alkenyl group or moiety is a linear or branched alkenyl group or moiety containing from 2 to 6 carbon atoms, such as a C₂-C₄ alkenyl group or moiety, for example ethenyl, propenyl, butenyl, or —CH₂—CH═C(CH₃)₂. A preferred alkenyl group is propenyl. Typically, an alkenyl group or moiety is saturated except for one double bond. A divalent alkenyl moiety (or alkenylene moiety) can be attached via the same carbon atoms, via adjacent carbon atoms or via non-adjacent carbon atoms.

As used herein, a C₂-C₆ alkynyl group or moiety is a linear or branched alkynyl group or moiety containing from 2 to 6 carbon atoms, such as a C₂-C₄ alkynyl group or moiety, for example ethynyl, propynyl and butynyl. Typically, an alkynyl group or moiety is saturated except for one triple bond. A divalent alkynyl moiety (or alkynylene moiety) can be attached via the same carbon atom, via adjacent carbon atoms or via non-adjacent carbon atoms.

When a phenyl moiety is fused to a cyclic group, it is preferably fused to a further phenyl ring to form a napthyl group.

As used herein, a 5- to 10-membered heteroaryl group is a monocyclic 5- to 10-membered aromatic ring, such as a 5- or 6-membered ring, containing at least one heteroatom, for example 1, 2 or 3 heteroatoms, selected from O, S and N. Examples Maude pyridyl, pyrazinyl, pyrimidinyl, pyridazinyl, furanyl, thienyl, imidazolyl, pyrrolyl, triazolyl, oxadiazolyl, oxazolyl, isoxazyl, thiadiazolyl, isothiazolyl, thiazolyl and pyrazolyl groups. Pyridyl, thienyl, pyrrolyl, pyrazolyl, thiazolyl and imidazolyl groups are preferred.

When a 5- to 10-membered heteroaryl moiety is fused to a phenyl, 5- to 6-membered heteroaryl, C₅-C₆ carbocyclyl or 5- to 6-membered heterocyclyl group, it is preferably a 5- to 6-membered heteroaryl moiety fused to a phenyl, 5- to 6-membered heteroaryl, C₅-C₆ carbocyclyl or 5- to 6-membered heterocyclyl group. When a 5- to 10-membered heteroaryl moiety is fused to a cyclic group, it is preferably fused to a phenyl group.

As used herein, a halogen is typically chlorine, fluorine, bromine or iodine and is preferably chlorine, fluorine or bromine.

As used herein, a C₁-C₂ haloalkyl group is typically a said C₁-C₂ alkyl group substituted by one or more said halogen atoms. Typically, it is substituted by 1, 2 or 3 said halogen atoms. Preferred haloalkyl groups include perhaloalkyl groups such as —CX₃ wherein X is a said halogen atom. A particularly preferred haloalkyl group is —CF₃.

As used herein, a C₃-C₆ carbocyclyl group or moiety is a monocyclic, non-aromatic saturated or unsaturated hydrocarbon ring, having from 3 to 6 carbon atoms. Preferably it is a saturated group, i.e. a C₃-C₆ cycloalkyl group. Examples include cyclopropyl, cyclobutyl, cyclopentyl and cyclohexyl. Preferred C₃-C₆ carbocyclyl groups or moieties are cyclopropyl, cyclopentyl and cyclohexyl.

When a C₃-C₆ carbocyclyl moiety is fused to a phenyl, 5- to 6-membered heteroaryl, C₅-C₆ carbocyclyl or 5- to 6-membered heterocyclyl group, it is preferably a C₅-C₆ carbocyclyl moiety fused to a phenyl, 5- to 6-membered heteroaryl, C₅-C₆ carbocyclyl or 5- to 6-membered heterocyclyl group. When a C₃-C₆ carbocyclyl moiety is fused to a cyclic group, it is preferably fused to a phenyl group. Examples of such fused groups include a cyclopentyl moiety that is fused to a phenyl group to form a dihydroindenyl group and a cyclohexyl group that is fused to a phenyl group to, form a tetrahydronaphthalenyl group.

As used herein, a 5- to 10-membered heterocyclyl group or moiety is a monocyclic, non-aromatic, saturated or unsaturated C₅-C₁₀ carbocyclic ring in which one or more, for example 1, 2 or 3, of the carbon atoms are replaced by a moiety selected from N, O, S, C(O), S(O) and S(O)₂. Preferably, only one or two carbon atoms are replaced with a —C(O)—, —S(O)— or —S(O)₂— moiety. More preferably, a 5- to 10-membered heterocyclyl group or moiety is a monocyclic, non-aromatic, saturated or unsaturated C₅-C₁₀ carbocyclic ring in which one or more, for example 1, 2 or 3, of the carbon atoms are replaced by a heteroatom selected from N, O and S.

Saturated heterocyclyl groups are preferred. Examples of suitable heterocyclyl groups include piperidinyl, piperazinyl, tetrahydropyranyl, dioxanyl, tetrahydrothiopyranyl, dithianyl, morpholinyl, thiomorpholinyl, S-oxo-thiomorpholino, S,S-dioxo-thiomorpholino, pyrrolidinyl, imidazolidinyl, pyrazolidinyl, tetrahydrofuranyl, dioxolanyl, tetrahydrothiophenyl, dithiolanyl, thiazolidinyl, oxazolidinyl, pyrrolidinonyl and pyrrolidine-2,5-dionyl groups. Preferred heterocyclyl groups are tetrahydrofuranyl, pyrrolidinyl, tetrahydropyranyl, morpholinyl, pyrrolidinonyl, pyrrolidine-2,5-dionyl and piperidinyl groups. Examples of preferred heterocyclyl groups are pyrrolidine-2,5-dionyl and piperidinyl groups.

When a 5- to 10-membered heterocyclyl moiety is fused to a phenyl, 5- to 6-membered heteroaryl, C₅-C₆ carbocyclyl or 5- to 6-membered heterocyclyl group, it is preferably a 5- to 6-membered heterocyclyl moiety fused to a phenyl, 5- to 6-membered heteroaryl, C₅-C₆ carbocyclyl or 5- to 6-membered heterocyclyl group. When a 5- to 10-membered heterocyclyl moiety is fused to a cyclic group, it is preferably fused to a phenyl group. Examples of such fused groups include a piperidinyl moiety that is fused to a phenyl group to form a tetrahydroisoquinolinyl group and a pyrrolidine-2,5-dionyl moiety that is fused to a phenyl group to form an isoindoline-1,3-dionyl group.

Typically, R¹ represents hydrogen or C₁-C₂ alkyl. Preferably, R¹ represents hydrogen.

Typically, Het represents —O—, —S— or —NH— or —N-Me-. Preferably, Het represents —O—.

Typically, X represents —CO—, —CO—O—, —CO—S— or —CONR¹—, wherein R¹ is as defined above. Preferably, X represents —CO—.

Typically, L represents a bond or a C₁-C₆ alkyl or C₂-C₆ alkenyl moiety. Preferably, L represents a bond or a C₁-C₄ alkyl moiety.

Typically, L′ represents a C₁-C₆ alkyl or C₂-C₆ alkenyl moiety. Preferably, L′ represents a C₁-C₄ alkyl or C₂-C₄ alkenyl moiety. Typically, the phenyl, heteroaryl, heterocyclyl and carbocyclyl groups and moieties in the groups R¹, R² and that formed by R² and R³ are unsubstituted or are substituted by one, two or three substituents which are the same or different and are selected from fluorine, chlorine, bromine, hydroxy, amino, thio, C₁-C₄ alkyl, C₂-C₄ alkenyl, cyano or -Het-L′, wherein Het and L′ are as defined above, the alkyl, alkenyl and alkynyl substituents being unsubstituted or substituted by one, two or three further substituents which are the same or different and are selected from fluorine, chlorine, bromine, hydroxy, amino and thio substituents.

Preferably, the phenyl, heteroaryl, heterocyclyl and carbocyclyl groups and moieties in the groups R¹, R² and that formed by R² and R³ are unsubstituted or are substituted by one, two or three unsubstituted substituents which are the same or different and are selected from fluorine, chlorine, bromine, C₁-C₄ alkyl, C₂-C₄ alkenyl, C₁-C₂ haloalkyl, —O—(C₁-C₄ alkyl), —O—(C₁-C₄ alkenyl) or —O—(C₁-C₂ haloalkyl) or by a single cyano or hydroxy group. Typically, when a phenyl, heteroaryl, heterocyclyl and carbocyclyl group or moiety is substituted by either cyano or nitro, each cyclic group or moiety only carries a single cyano or nitro group.

Typically, the alkyl, alkenyl and alkynyl groups and moieties in R¹ to R⁵ are unsubstituted or substituted by one, two or three fluorine or chlorine substituents. Preferably, the alkyl, alkenyl and alkynyl groups and moieties in R¹ to R⁵ are unsubstituted or substituted by one, two or three fluorine substituents. More preferably, the alkyl, alkenyl and alkynyl groups and moieties in R¹ to R⁵ are unsubstituted.

Typically, A represents a phenyl, 5- to 6-membered heteroaryl, C₃-C₆ carbocyclyl or 5- to 6-membered heterocyclyl group, said group being optionally fused to a phenyl, 5- to 6-membered heteroaryl, C₅-C₆ carbocyclyl or 5- to 6-membered heterocyclyl moiety. Preferably, A represents a phenyl, 5- to 6-membered heteroaryl, C₃-C₆ carbocyclyl or 5- to 6-membered heterocyclyl group, said group being optionally fused to a phenyl moiety. More preferably, A represents a phenyl, cyclopropyl, dihydroindenyl, tetrahydronaphthalenyl, pyridyl or piperidinyl group.

Typically, when R¹ comprises a group A, A is a phenyl, 5- to 6-membered heteroaryl, C₃-C₆ carbocyclyl or 5- to 6-membered heterocyclyl group. Preferably, when R¹ comprises a group A, A is a phenyl or pyridyl group. Typically, when R¹ comprises a group A which is a cyclic moiety fused to a further cyclic moiety, R¹ comprises only one such fused group.

Typically, when R² comprises a group A, A is a phenyl, 5- to 6-membered heteroaryl, C₃-C₆ carbocyclyl or 5- to 6-membered heterocyclyl group. Preferably, when R² comprises a group A, A is a phenyl, cyclopropyl, dihydroindenyl, tetrahydronaphthalenyl, or piperidinyl group. Typically, when R² comprises a group A which is a cyclic moiety fused to a further cyclic moiety, R² comprises only one such fused group.

Typically, A′ represents -Het-A or —X-A, wherein Het is —O—, X is —C(O)— and A is as defined above. Preferably, A′ represents —O-phenyl or —C(O)-phenyl.

Typically, Z is -Het-L′- or —X-L′-, wherein Het, X and L′ are as defined above. Preferably, Z is —O—(C₁-C₂

Typically, R is hydrogen or C₁-C₂ alkyl. Preferably, R is hydrogen.

Typically, when R¹ represents -L-A, L is a bond or a C₁-C₂ alkyl moiety and A is a phenyl, 5- to 6-membered heteroaryl, C₃-C₆ carbocyclyl or 5- to 6-membered heterocyclyl group. Preferably, when R¹ represents -L-A, L is a bond or a C₁-C₂ alkyl moiety and A is a phenyl group.

Typically, when R¹ represents -L-CR(A)(L-A), each L is the same or different and is a bond or a C₁-C₂ alkyl moiety, R is hydrogen or C₁-C₂ alkyl and each A is the same or different and is a phenyl, 5- to 6-membered heteroaryl, C₃-C₆ carbocyclyl or 5- to 6-membered heterocyclyl group. Preferably, when R¹ represents -L-CR(A)(L-A), each L represents a bond, R is hydrogen and A is a phenyl group.

Typically, when R¹ represents -L-A-A′, L is a bond or a C₁-C₂ alkyl moiety, A′ is -Het-A or —X-A wherein Het and X are as defined above and each A is the same or different and is a phenyl, 5- to 6-membered heteroaryl, C₃-C₆ carbocyclyl or 5- to 6-membered heterocyclyl group. Preferably, when R¹ represents -L-A-A′, L is a bond or a methylene moiety, A′ is —O-A or —C(O)-A and each A is the same or different and is a phenyl or pyridyl group.

Typically, when R¹ represents -A-Z-A, each A is the same or different and is a phenyl, 5- to 6-membered heteroaryl, C₃-C₆ carbocyclyl or 5- to 6-membered heterocyclyl group and Z is -Het-L′- or —X-L′-, wherein Het, X and L′ are as defined above. Preferably, when R¹ represents -A-Z-A, each A is a phenyl group and Z is —O—C₁-C₂ alkyl-.

Typically, R¹ represents:

(a) -L-A wherein L and A are as defined above;

(b) -L-CR(A)(L-A) wherein R is as defined above, each L is the same or different and is as defined above and each A is the same or different and is as defined above;

(c) -L-A-A′ wherein L, A and A′ are as defined above; or

(d) -A-Z-A wherein Z is as defined above and each A is the same or different and is as defined above; or

Preferably, R¹ represents:

(a) -L-A wherein L is a bond or a C₁-C₂ alkyl moiety and A is a phenyl group;

(b) —CHA₂ wherein A is a phenyl group;

(c) -L-A-A′ wherein L is a bond or a methylene moiety, A′ is —O-A or —C(O)-A and each A is the same or different and is a phenyl or pyridyl group; or

(d) -A-Z-A wherein each A is a phenyl group and Z is —O—C₁-C₂ alkyl-.

Typically, J represents —NR⁵—, —O— or a direct bond, wherein R⁵ is hydrogen or C₁-C₄ alkyl. Preferably, J is —NH—, —NMe- or a direct bond.

Typically, R⁴ is represents hydrogen or C₁-C₄ alkyl. Preferably, R⁴ is represents hydrogen or methyl.

Typically, L″ is —X-L′ or —CONH₂, wherein X and L′ are as defined above. Preferably, L″ is —X-L′ or —CONH₂, wherein X is —C(O)—O— and L′ is C₁-C₂ alkyl.

Typically, when R² represents -L-A, L is a bond or a C₁-C₄ alkyl moiety and A is a phenyl, 5- to 6-membered heteroaryl, C₃-C₆ carbocyclyl or 5- to 6-membered heterocyclyl group which can be optionally fused to a phenyl, 5- to 6-membered heteroaryl, C₅-C₆ carbocyclyl or 5- to 6-membered heterocyclyl group. Preferably, when R² represents -L-A, L is a bond or a C₁-C₄ alkyl moiety and A is a phenyl, dihydroindenyl or tetrahydronaphthalenyl group.

Typically, when R² represents -L-A′, L is a bond or a C₁-C₄ alkyl moiety, A′ is -Het-A or —X-A wherein Het and X are as defined above and A is a phenyl, 5- to 6-membered heteroaryl, C₃-C₆ carbocyclyl or 5- to 6-membered heterocyclyl group which can be optionally fused to a phenyl, 5- to 6-membered heteroaryl, C₅-C₆ carbocyclyl or 5- to 6-membered heterocyclyl group. Preferably, when R² represents -L-A′, L is a bond and A′ is —(CO)-phenyl.

Typically, when R² represents -L-A-A′, L is a bond or a C₁-C₄ alkyl moiety, A′ is -Het-A or —X-A wherein Het and X are as defined above and each A is the same or different and is a phenyl, 5- to 6-membered heteroaryl, C₃-C₆ carbocyclyl or 5- to 6-membered heterocyclyl group. Preferably, when R³ represents -L-A-A′, L is a C₁-C₂ alkyl moiety, A′ is —O-A and each A is a phenyl group. Typically, when R² represents -L-A-L-A, each L is the same or different and is a bond or a C₁-C₄ alkyl moiety and each A is the same or different and is a phenyl, 5- to 6-membered heteroaryl, C₃-C₆ carbocyclyl or 5- to 6-membered heterocyclyl group. Preferably, when R² represents -L-A-L-A, each L is the same or different and is a bond or a C₁-C₂ alkyl moiety and each A is the same or different and is a phenyl or piperidinyl group.

Typically, when R² represents -L-CR(A)(L-A), each L is the same or different and is a bond or a C₁-C₂ alkyl moiety, each A is the same or different and is a phenyl, 5- to 6-membered heteroaryl, C₃-C₆ carbocyclyl or 5- to 6-membered heterocyclyl group and R is hydrogen or C₁-C₂ alkyl. Preferably, when R² represents -L-CR(A)(L-A), each L represents a bond, each A is the same or different and is a phenyl or cyclopropyl group and R is hydrogen. In a preferred embodiment, when R² represents -L-CR(A)(L-A), the moiety (L-A) is (L-phenyl).

Typically, when R² represents -L-CR(A)(L″), L is a bond or a C₁-C₂ alkyl moiety, A is a phenyl, 5- to 6-membered heteroaryl, C₃-C₆ carbocyclyl or 5- to 6-membered heterocyclyl group, R is hydrogen or C₁-C₂ alkyl and L″ is as defined above. Preferably, when R² represents -L-CR(A)(L″), L is a bond, A is a phenyl group, R is hydrogen and L″ is —X-L′ or —CONH₂, wherein X is —C(O)—O— and L′ is C₁-C₂ alkyl.

Typically, R² represents -L-A, -L-A′, -L-A-A′, -L-A-L-A, -L-CR(A)(L-A) or -L-CR(A)(L″) wherein L′, L″, A′ and R are as defined above, each L is the same or different and is as defined above and each A is the same or different and is as defined above.

Preferably, R² represents -L-A wherein L is a bond or a C₁-C₄ alkyl moiety and A is a phenyl, dihydroindenyl or tetrahydronaphthalenyl group; -L-A′ wherein L is a bond and A′ is —(CO)-phenyl; -L-A-A′ wherein L is a C₁-C₂ alkyl moiety, A′ is —O-A and each A is a phenyl group; -L-A-L-A wherein each L is a bond or a C₁-C₂ alkyl moiety and each A is the same or different and is a phenyl or piperidinyl group; -L-CR(A)(L-phenyl) wherein each L is a bond, A is a phenyl or cyclopropyl group and R is hydrogen; or -L-CR(A)(L″) wherein L is a bond, A is a phenyl group, R is hydrogen and L″ is —X-L′ or —CONH₂, wherein X is —C(O)—O— and L′ is C₁-C₂ alkyl.

Typically, R³ represents hydrogen, C₁-C₆ alkyl, C₂-C₆ alkenyl or —(CO)-L′, wherein L′ is as defined above. Preferably, R³ represents hydrogen, C₁-C₂ alkyl or —(CO)-L′, wherein L′ is C₁-C₂ haloalkyl.

Typically, when R² and R³ form, together with the nitrogen to which they are attached, a 5- to 10-membered heteroaryl or 5- to 10-membered heterocyclyl ring, they form a 5- to 6-membered heteroaryl or 5- to 6-membered heterocyclyl ring which can be optionally fused to a phenyl, 5- to 6-membered heteroaryl, C₃-C₅ carbocyclyl or 5- to 6-membered heterocyclyl group. In a preferred embodiment, the ring formed by R² and R³ is fused to a phenyl group. Preferably, when R² and R³ form, together with the nitrogen to which they are attached, a 5- to 10-membered heteroaryl or 5- to 10-membered heterocyclyl ring, they form an tetrahydroisoquinolinyl or isoindoline-1,3-dionyl group.

Preferred compounds of formula (I) are those wherein:

R¹ represents:

(a) -L-A wherein L represents a bond, C₁-C₆ alkyl or C₂-C₆ alkenyl moiety and A represents a phenyl, 5- to 6-membered heteroaryl, C₃-C₆ carbocyclyl or 5- to 6-membered heterocyclyl group, said group being optionally fused to a phenyl, 5- to 6-membered heteroaryl, C₅-C₆ carbocyclyl or 5- to 6-membered heterocyclyl moiety;

(b) -L-CR(A)(L-A) wherein R is hydrogen or C₁-C₂ alkyl, each L is the same or different and is as defined above and each A is the same or different and is as defined above;

(c) -L-A-A′ wherein A′ represents -Het-A or —X-A wherein Het represents —O—, —S— or —NR¹—, X represents —CO—, —CO—O—, —CO—S— or —CONR¹—, wherein R′ represents hydrogen or C₁-C₂ alkyl and wherein L is as defined above and each A is the same or different and is as defined above; or

(d) -A-Z-A wherein Z is -Het-L′- or —X-L′-, wherein L′ represents a C₁-C₆ alkyl or C₂-C₆ alkenyl moiety, Het and X are as defined above and each A is the same or different and is as defined above;

J represents —NR⁵—, —O— or a direct bond wherein R⁵ represents hydrogen or C₁-C₄ alkyl;

R⁴ represents hydrogen or C₁-C₄ alkyl; and either —R² represents -L-A, -L-A′, -L-A-A′, -L-A-L-A, -L-CR(A)(L-A) or -L-CR(A)(L″) wherein L″ is —X-L′ or —CONH₂ and wherein X, L′, A′ and R are as defined above, each L is the same or different and is as defined above and each A is the same or different and is as defined above, and

R³ represents hydrogen, C₁-C₆ alkyl, C₂-C₆ alkenyl or —(CO)-L′, wherein L′ is as defined above; or

R² and R³ form, together with the nitrogen to which they are attached, a 5- to 6-membered heteroaryl or 5- to 6-membered heterocyclyl ring which can be optionally fused to a phenyl, 5- to 6-membered heteroaryl, C₃-C₅ carbocyclyl or 5- to 6-membered heterocyclyl group,

wherein:

the phenyl, heteroaryl, heterocyclyl and carbocyclyl groups and moieties in the groups R¹, R² and that formed by R² and R³ are unsubstituted or are substituted by one, two or three substituents which are the same or different and are selected from fluorine, chlorine, bromine, hydroxy, amino, thio, C₁-C₄ alkyl, C₂-C₄ alkenyl, cyano or -Het-L′, wherein Het and L′ are as defined above; and

the alkyl, alkenyl and alkynyl groups and moieties in R¹ to R⁵ are unsubstituted or substituted by one, two or three substituents which are the same or different and are selected from fluorine, chlorine, bromine, hydroxy, amino and thio substituents.

More preferred compounds of formula (I) are those wherein:

R¹ represents:

(a) -L-A wherein L is a bond or a C₁-C₂ alkyl moiety and A is a phenyl group;

(b) —CH(A)₂ wherein A is a phenyl group;

(c) -L-A-A′ wherein L is a bond or a methylene moiety, A′ is —O-A or —C(O)-A and each A is the same or different and is a phenyl or pyridyl group; or

(d) -A-Z-A wherein each A is a phenyl group and Z is —O—C₁-C₂ alkyl-;

J is —NH—, —NMe- or a direct bond;

R⁴ is represents hydrogen or methyl; and either

R² represents -L-A wherein L is a bond or a C₁-C₄ alkyl moiety and A is a phenyl, dihydroindenyl or tetrahydronaphthalenyl group; -L-A′ wherein L is a bond and A′ is —(CO)-phenyl; -L-A-A′ wherein L is a C₁-C₂ alkyl moiety, A′ is —O-A and each A is a phenyl group; -L-A-L-A wherein each L is a bond or a C₁-C₂ alkyl moiety and each A is the same or different and is a phenyl or piperidinyl group; -L-CR(A)(L-phenyl) wherein each L is a bond, A is a phenyl or cyclopropyl group and R is hydrogen; or -L-CR(A)(L″) wherein L is a bond, A is a phenyl group, R is hydrogen and L″ is —X-L′ or —CONH₂, wherein X is —C(O)—O— and L′ is C₁-C₂ alkyl; and

R³ represents hydrogen, C₁-C₂ alkyl or —(CO)-L′, wherein L′ is C₁-C₂ haloalkyl; or

R² and R³ form, together with the nitrogen to which they are attached, a 5- to 10-membered heteroaryl or 5- to 10-membered heterocyclyl ring, they form an tetrahydroisoquinolinyl or isoindoline-1,3-dionyl group,

wherein:

the phenyl, heteroaryl, heterocyclyl and carbocyclyl groups and moieties in the groups R¹, R² and that formed by R² and R³ are unsubstituted or are substituted by one, two or three unsubstituted substituents which are the same or different and are selected from fluorine, chlorine, bromine, C₁-C₄ alkyl, C₂-C₄ alkenyl, C₁-C₂ haloalkyl, —O—(C₁-C₄ alkyl), —O—(C₁-C₄ alkenyl) or —O—(C₁-C₂ haloalkyl) or by a single cyano or hydroxy group; and

the alkyl, alkenyl and alkynyl groups and moieties in R¹ to R⁴ are unsubstituted.

Examples of particularly preferred compounds of the present invention are:
3-(1-Methyl-2-o-tolyl-ethylamino)-azetidine-1-carboxylic acid [1-(4-trifluoromethyl-phenyl)-ethyl]-amide;
3-[1-(4-Fluoro-phenyl)-ethylamino]-azetidine-1-carboxylic acid [bis-(4-fluoro-phenyl)-methyl]-amide;
3-(Indan-2-ylamino)-azetidine-1-carboxylic acid [1-(4-trifluoromethyl-phenyl)-ethyl]-amide;
3-(1,2,3,4-Tetrahydro-naphthalen-2-ylamino)-azetidine-1-carboxylic acid [144-trifluoromethyl-phenyl)-ethyl]-amide;
3-(6-Fluoro-1,2,3,4-tetrahydro-naphthalen-2-ylamino)-azetidine-1-carboxylic acid [1-(4-trifluoromethyl-phenyl)-ethyl]-amide;
3-(5-Fluoro-indan-1-ylamino)-azetidine-1-carboxylic acid (4-butoxy-phenyl)-amide;
3-(5-Fluoro-indan-1-ylamino)-azetidine-1-carboxylic acid [4-(4-fluoro-phenoxy)-phenyl]-amide;
3-(6-Fluoro-1,2,3,4-tetrahydro-naphthalen-2-ylamino)-azetidine-1-carboxylic acid [4-(4-fluoro-phenoxy)-phenyl]-amide;
3-(1,2,3,4-Tetrahydro-naphthalen-2-ylamino)-azetidine-1-carboxylic acid [4-(4-fluoro-phenoxy)-phenyl]-amide;
3-(5-Fluoro-indan-1-ylamino)-azetidine-1-carboxylic acid [4-(4-chloro-phenoxy)-phenyl]-amide;
3-(5-Fluoro-indan-1-ylamino)-azetidine-1-carboxylic acid [3-fluoro-4-(4-fluoro-phenoxy)-phenyl]-amide;
3-(5-Hydroxy-indan-1-ylamino)-azetidine-1-carboxylic acid [4-(4-fluoro-phenoxy)-phenyl]-amide;
3-(5-Fluoro-indan-1-ylamino)-azetidine-1-carboxylic acid [4-(3-fluoro-phenoxy)-phenyl]-amide;
3-(5-Fluoro-indan-1-ylamino)-azetidine-1-carboxylic acid [4-(2-fluoro-phenoxy)-phenyl]-amide;
3-(5-Chloro-indan-1-ylamino)-azetidine-1-carboxylic acid [3-fluoro-4-(4-fluoro-phenoxy)-phenyl]-amide;
3-(5-Bromo-indan-1-ylamino)-azetidine-1-carboxylic acid [3-fluoro-4-(4-fluoro-phenoxy)-phenyl]-amide;
3-(5-Bromo-indan-1-ylamino)-azetidine-1-carboxylic acid [4-(4-fluoro-phenoxy)-phenyl]-amide;
3-(5-Chloro-indan-1-ylamino)-azetidine-1-carboxylic acid [4-(4-fluoro-phenoxy)-phenyl]-amide;
3-(1,2,3,4-Tetrahydro-naphthalen-1-ylamino)-azetidine-1-carboxylic acid [4-(4-fluoro-phenoxy)-phenyl]-amide;
3-(5-Fluoro-indan-1-ylamino)-azetidine-1-carboxylic acid [4-(4-fluoro-phenoxy)-phenyl]-methyl-amide;
3-(5-Methoxy-indan-1-ylamino)-azetidine-1-carboxylic acid [4-(4-fluoro-phenoxy)-phenyl]-amide;
3-(5-Fluoro-indan-1-ylamino)-azetidine-1-carboxylic acid (4-trifluoromethyl-phenyl)-amide;
3-{[Cyclopropyl-(4-fluoro-phenyl)-methyl]-amino}-azetidine-1-carboxylic acid (4-trifluoromethyl-phenyl)-amide;
3-(5-Fluoro-indan-1-ylamino)-azetidine-1-carboxylic acid (3-trifluoromethyl-phenyl)-amide;
3-(2-Methyl-indan-1-ylamino)-azetidine-1-carboxylic acid (4-phenoxy-phenyl)-amide;
3-(3-Methyl-indan-1-ylamino)-azetidine-1-carboxylic acid (4-phenoxy-phenyl)-amide;
3-(5-Fluoro-indan-1-ylamino)-azetidine-1-carboxylic acid (4-phenoxy-phenyl)-amide;
3-(5-Fluoro-indan-1-ylamino)-azetidine-1-carboxylic acid (3-phenoxy-phenyl)-amide;
3-[(5-Fluoro-indan-1-yl)-methyl-amino]-azetidine-1-carboxylic acid (3-phenoxy-phenyl)-amide;
3-(Indan-1-ylamino)-azetidine-1-carboxylic acid (4-phenoxy-phenyl)-amide;
3-(5-Fluoro-indan-1-ylamino)-azetidine-1-carboxylic acid (4-benzoyl-phenyl)-amide;
3-(1,2,3,4-Tetrahydro-naphthalen-2-ylamino)-azetidine-1-carboxylic acid (4-trifluoromethyl-phenyl)-amide;
3-(5-Hydroxy-indan-1-ylamino)-azetidine-1-carboxylic acid (4-trifluoromethyl-phenyl)-amide;
3-(1,2,3,4-Tetrahydro-naphthalen-2-ylamino)-azetidine-1-carboxylic acid (4-phenoxy-phenyl)-amide;
3-(6-Fluoro-1,2,3,4-tetrahydro-naphthalen-2-ylamino)-azetidine-1-carboxylic acid (4-trifluoromethyl-phenyl)-amide;
3-(1,2,3,4-Tetrahydro-naphthalen-1-ylamino)-azetidine-1-carboxylic acid (4-trifluoromethyl-phenyl)-amide;
3-[(5-Fluoro-indan-1-yl)-methyl-amino]-azetidine-1-carboxylic acid [4-(4-fluoro-phenoxy)-phenyl]-amide;
3-[(5-Fluoro-indan-1-yl)-methyl-amino]azetidine-1-carboxylic acid (4-phenoxy-phenyl)-amide;
3-[(5-Fluoro-indan-1-yl)-methyl-amino]-azetidine-1-carboxylic acid (4-trifluoromethyl-phenyl)-amide;
3-[(5-Fluoro-indan-1-yl)-methyl-amino]azetidine-1-carboxylic acid (3-trifluoromethyl-phenyl)-amide;
3-(5-Fluoro-indan-1-ylamino)-3-methyl-azetidine-1-carboxylic acid [4-(4-fluoro-phenoxy)-phenyl]-amide;
3-Methyl-3-(1,2,3,4-tetrahydro-naphthalen-2-ylamino)-azetidine-1-carboxylic acid (4-trifluoromethyl-phenyl)-amide;
[3-(5-Fluoro-indan-1-ylamino)-azetidin-1-yl]-[4-(4-fluoro-phenoxy)-phenyl]-methanone;
(4-Phenoxy-phenyl)-[3-(1-phenyl-ethylamino)-azetidin-1-yl]-methanone;
1-[3-(Indan-2-ylamino)-azetidin-1-yl]-2-(4-phenoxy-phenyl)-ethanone;
2-(4-Phenoxy-phenyl)-1-[3-(1-phenyl-ethylamino)-azetidin-1-yl]-ethanone;
1-[3-(5-Fluoro-indan-1-ylamino)-azetidin-1-yl]-2-(4-phenoxy-phenyl)-ethanone;
1-[3-(5-Fluoro-indan-1-ylamino)-azetidin-1-yl]-2-[4-(4-fluoro-phenoxy)-phenyl]-ethanone;
2-[4-(4-Fluoro-phenoxy)-phenyl]-1-[3-(indan-2-ylamino)-azetidin-1-yl]-ethanone;
1-[3-(5-Fluoro-indan-2-ylamino)-azetidin-1-yl]-2-[4-(4-fluoro-phenoxy)-phenyl]-ethanone;
3-(2-Allyloxy-benzylamino)-azetidine-1-carboxylic acid (4-benzyloxy-phenyl)-amide;
3-(2,6-Difluoro-benzylamino)-azetidine-1-carboxylic acid (4-benzyloxy-phenyl)-amide;
[1-(4-Phenoxy-phenylcarbamoyl)-azetidin-3-ylamino]-phenyl-acetic acid methyl ester;
3-[1-(4-Methoxy-phenyl)-ethylamino]-azetidine-1-carboxylic acid (4-phenoxy-phenyl)-amide;
3-[(Carbamoyl-phenyl-methyl)-amino]-azetidine-1-carboxylic acid (4-phenoxy-phenyl)-amide;
3-[1-(4-Fluoro-phenyl)-ethylamino]-azetidine-1-carboxylic acid (4-trifluoromethyl-phenyl)-amide;
3-[1-(2-Chloro-4-fluoro-phenyl)-ethylamino]-azetidine-1-carboxylic acid (4-phenoxy-phenyl)-amide;
3-[1-(2-Chloro-4-fluoro-phenyl)-ethylamino]-azetidine-1-carboxylic acid (3-phenoxy-phenyl)-amide;
3-{[1-(2-Chloro-4-fluoro-phenyl)-ethyl]-methyl-amino}-azetidine-1-carboxylic acid (4-trifluoromethyl-phenyl)-amide;
3-{[1-(2-Chloro-4-fluoro-phenyl)-ethyl]-methyl-amino}-azetidine-1-carboxylic acid (4-phenoxy-phenyl)-amide;
3-(1-o-Tolyl-ethylamino)-azetidine-1-carboxylic acid (4-phenoxy-phenyl)-amide;
3-[Methyl-(1-o-tolyl-ethyl)-amino]-azetidine-1-carboxylic acid (4-phenoxy-phenyl)-amide;
3-[1-(2-Chloro-phenyl)-ethylamino]-azetidine-1-carboxylic acid (4-phenoxy-phenyl)-amide;
3-(8-Methoxy-3,4-dihydro-1H-isoquinolin-2-yl)-azetidine-1-carboxylic acid (4-trifluoromethyl-phenyl)-amide;
3-(1-o-Tolyl-ethylamino)-azetidine-1-carboxylic acid (4-butoxy-phenyl)-amide;
3-[1-(4-Fluoro-phenyl)-ethylamino]-azetidine-1-carboxylic acid (4-butoxy-phenyl)-amide;
3-{[1-(2-Chloro-phenyl)-ethyl]-methyl-amino}-azetidine-1-carboxylic acid (4-phenoxy-phenyl)-amide;
3-(1-o-Tolyl-ethylamino)-azetidine-1-carboxylic acid (3-butoxy-phenyl)-amide;
3-[1-(4-Fluoro-phenyl)-ethylamino]-azetidine-1-carboxylic acid (3-butoxy-phenyl)-amide;
3-[1-(4-Trifluoromethoxy-phenyl)-ethylamino]-azetidine-1-carboxylic acid (4-phenoxy-phenyl)-amide;
3-[1-(2-Chloro-phenyl)-ethylamino]-azetidine-1-carboxylic acid (4-butoxy-phenyl)-amide;
3-[1-(4-Fluoro-phenyl)-ethylamino]-azetidine-1-carboxylic acid (4-trifluoromethoxy-phenyl)-amide;
3-[1-(4-Fluoro-phenyl)-ethylamino]-azetidine-1-carboxylic acid (3-trifluoromethyl-phenyl)-amide;
3-[1-(2-Chloro-phenyl)-ethylamino]-azetidine-1-carboxylic acid (3-trifluoromethyl-phenyl)-amide;
3-(1-o-Tolyl-ethylamino)-azetidine-1-carboxylic acid (3-trifluoromethyl-phenyl)-amide;
3-(8-Methoxy-3,4-dihydro-1H-isoquinolin-2-yl)-azetidine-1-carboxylic acid (4-butoxy-phenyl)-amide;
3-[1-(4-Phenoxy-phenyl)-ethylamino]-azetidine-1-carboxylic acid (4-phenoxy-phenyl)-amide;
3-[1-(4-Phenoxy-phenyl)-ethylamino]-azetidine-1-carboxylic acid (4-trifluoromethyl-phenyl)-amide;
3-[1-(4-Fluoro-phenyl)-ethylamino]-azetidine-1-carboxylic acid (4-phenoxy-phenyl)-amide;
3-{Methyl-[1-(4-trifluoromethoxy-phenyl)-ethyl]-amino}-azetidine-1-carboxylic acid (4-phenoxy-phenyl)-amide;
3-{[1-(4-Fluoro-phenyl)-ethyl]-methyl-amino}-azetidine-1-carboxylic acid (2-fluoro-5-trifluoromethyl-phenyl)-amide;
3-{[1-(4-Fluoro-phenyl)-ethyl]-methyl-amino}-azetidine-1-carboxylic acid (3-fluoro-5-trifluoromethyl-phenyl)-amide;
3-{[1-(4-Fluoro-phenyl)-ethyl]-methyl-amino}-azetidine-1-carboxylic acid (4-fluoro-3-trifluoromethyl-phenyl)-amide;
3-{[1-(4-Fluoro-phenyl)-ethyl]-methyl-amino}-azetidine-1-carboxylic acid (2-fluoro-3-trifluoromethyl-phenyl)-amide;
3-{[1-(4-Fluoro-phenyl)-ethyl]-methyl-amino}-azetidine-1-carboxylic acid (3-butoxy-phenyl)-amide;
3-{[1-(4-Fluoro-phenyl)-ethyl]-methyl-amino}-azetidine-1-carboxylic acid (4-butoxy-phenyl)-amide;
3-[Methyl-(1-o-tolyl-ethyl)-amino]-azetidine-1-carboxylic acid (3-butoxy-phenyl)-amide;
3-[1-(2-Trifluoromethyl-phenyl)-ethylamino]-azetidine-1-carboxylic acid (4-phenoxy-phenyl)-amide;
3-[Methyl-(1-o-tolyl-ethyl)-amino]-azetidine-1-carboxylic acid (4-butoxy-phenyl)-amide;
3-{Methyl-[1-(4-phenoxy-phenyl)-ethyl]-amino}-azetidine-1-carboxylic acid (4-trifluoromethyl-phenyl)-amide;
3-{[1-(4-Fluoro-phenyl)-ethyl]-methyl-amino}-azetidine-1-carboxylic acid (4-phenoxy-phenyl)-amide;
3-[[1-(2-Chloro-4-fluoro-phenyl)-ethyl]-(2,2,2-trifluoro-acetyl)-amino]-azetidine-1 carboxylic acid (4-phenoxy-phenyl)-amide;
3-{Methyl-[1-(2-trifluoromethyl-phenyl)-ethyl]-amino}-azetidine-1-carboxylic acid (4-phenoxy-phenyl)-amide;
3-[1-(2-Chloro-phenyl)-ethylamino]-azetidine-1-carboxylic acid [4-(4-fluoro-phenoxy)-phenyl]-amide;
3-{[1-(4-Fluoro-phenyl)ethyl]-methyl-amino}-azetidine-1-carboxylic acid (3-trifluoromethyl-phenyl)-amide;
3-[Methyl-(1-o-tolyl-ethyl)-amino]-azetidine-1-carboxylic acid (3-trifluoromethyl-phenyl)-amide;
3-{[1-(2-Chloro-phenyl)-ethyl]-methyl-amino}-azetidine-1-carboxylic acid (3-trifluoromethyl-phenyl)-amide;
3-{[1-(4-Fluoro-phenyl)-ethyl]-methyl-amino}-azetidine-1-carboxylic acid (4-trifluoromethoxy-phenyl)-amide;
3-[Methyl-(1-methyl-1-phenyl-ethyl)-amino]-azetidine-1-carboxylic acid (4-phenoxy-phenyl)-amide;
3-{[1-(2-Chloro-4-fluoro-phenyl)-ethyl]-methyl-amino}-azetidine-1-carboxylic acid (3-phenoxy-phenyl)-amide;
3-[1-(2,4-Dimethyl-phenyl)-ethylamino]-azetidine-1-carboxylic acid (4-phenoxy-phenyl)-amide;
3-[1-(2-Chloro-phenyl)-ethylamino]-azetidine-1-carboxylic acid (3-phenoxy-phenyl)-amide;
3-[1-(4-Fluoro-phenyl)-ethylamino]-azetidine-1-carboxylic acid (3-phenoxy-phenyl)-amide;
3-(1-o-Tolyl-ethylamino)-azetidine-1-carboxylic acid (4-trifluoromethyl-phenyl)-amide;
3-{[1-(2,4-Dimethyl-phenyl)-ethyl]-methyl-amino}-azetidine-1-carboxylic acid (4-phenoxy-phenyl)-amide;
3-(1-o-Tolyl-ethylamino)-azetidine-1-carboxylic acid (3-phenoxy-phenyl)-amide;
3-[Methyl-(1-o-tolyl-ethyl)-amino]-azetidine-1-carboxylic acid (3-phenoxy-phenyl)-amide;
3-(1-o-Tolyl-ethylamino)-azetidine-1-carboxylic acid [4-(4-fluoro-phenoxy)-phenyl]-amide;
3-{[1-(4-Fluoro-phenyl)-ethyl]-methyl-amino}-azetidine-1-carboxylic acid (3-phenoxy-phenyl)-amide;
3-[Methyl-(1-o-tolyl-ethyl)-amino]-azetidine-1-carboxylic acid (4-trifluoromethyl-phenyl)-amide;
3-[1-(4-Fluoro-2-methyl-phenyl)-ethylamino]-azetidine-1-carboxylic acid (4-phenoxy-phenyl)-amide;
3-[1-(4-Fluoro-2-methyl-phenyl)-ethylamino]-azetidine-1-carboxylic acid (3-phenoxy-phenyl)-amide;
3-[1-(4-Fluoro-2-methyl-phenyl)-ethylamino]-azetidine-1-carboxylic acid (3-trifluoromethyl-phenyl)-amide;
3-[1-(4-Fluoro-2-methyl-phenyl)-ethylamino]-azetidine-1-carboxylic acid (4-trifluoromethyl-phenyl)-amide;
3-[1-(4-Methoxy-phenyl)-ethylamino]-azetidine-1-carboxylic acid (4-trifluoromethyl-phenyl)-amide;
3-(5-Fluoro-1,3-dioxo-1,3-dihydro-isoindol-2-yl)-azetidine-1-carboxylic acid (4-phenoxy-phenyl)-amide;
3-{[1-(4-Fluoro-2-methyl-phenyl)-ethyl]-methyl-amino}-azetidine-1-carboxylic acid (4-phenoxy-phenyl)-amide;
3-(1-m-Tolyl-ethylamino)-azetidine-1-carboxylic acid (4-phenoxy-phenyl)-amide;
3-[1-(4-Fluoro-2-methyl-phenyl)-ethylamino]-azetidine-1-carboxylic acid [4-(4-fluoro-phenoxy)-phenyl]-amide;
3-[1-(4-Piperidin-1-yl-phenyl)-ethylamino]-azetidine-1-carboxylic acid (4-phenoxy-phenyl)-amide;
3-(1-p-Tolyl-ethylamino)-azetidine-1-carboxylic acid (4-phenoxy-phenyl)-amide;
3-(1-p-Tolyl-ethylamino)-azetidine-1-carboxylic acid (4-trifluoromethyl-phenyl)-amide;
3-(2,3-Dihydro-benzofuran-3-ylamino)-azetidine-1-carboxylic acid (4-phenoxy-phenyl)-amide;
3-[1-(2-Chloro-phenyl)-ethylamino]-azetidine-1-carboxylic acid (4-trifluoromethyl-phenyl)-amide;
3-(5-Fluoro-1,3-dioxo-1,3-dihydro-isoindol-2-yl)-azetidine-1-carboxylic acid (4-trifluoromethyl-phenyl)-amide;
3-[1-(4-Cyano-phenyl)-ethylamino]-azetidine-1-carboxylic acid (4-trifluoromethyl-phenyl)-amide;
3-[1-(4-Cyano-phenyl)-ethylamino]-azetidine-1-carboxylic acid [4-(4-fluoro-phenoxy)-phenyl]-amide;
3-(2,6-Difluoro-benzylamino)-azetidine-1-carboxylic acid (4-phenoxy-phenyl)-amide;
3-(2,6-Difluoro-benzylamino)-azetidine-1-carboxylic acid [4-(4-fluoro-phenoxy)-phenyl]-amide;
3-[1-(2,6-Difluoro-phenyl)-ethylamino]-azetidine-1-carboxylic acid (4-phenoxy-phenyl)-amide;
3-[1-(2,6-Difluoro-phenyl)-ethylamino]-azetidine-1-carboxylic acid [4-(4-fluoro-phenoxy)-phenyl]-amide;
3-(2-Methyl-benzoylamino)-azetidine-1-carboxylic acid (4-phenoxy-phenyl)-amide;
3-(5-Chloro-indan-1-ylamino)-azetidine-1-carboxylic acid [4-(2-fluoro-phenoxy)-phenyl]-amide; and
3-(5-Methoxy-indan-1-ylamino)-azetidine-1-carboxylic acid [4-(3-fluoro-phenoxy)-phenyl]-amide,

and pharmaceutically acceptable salts thereof.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulfonic, ethanesulfonic, benzenesulfonic or p-toluenesulfonic acid. Pharmaceutically acceptable bases include alkali metal (e.g. sodium or potassium) and alkali earth metal (e.g. calcium or magnesium) hydroxides and organic bases such as alkyl amines, aralkyl amines or heterocyclic amines.

The compounds of the invention can contain one or more chiral centres. For the avoidance of doubt, the chemical structures depicted herein are intended to embrace all stereoisomers of the compounds shown, including racemic and non-racemic mixtures and pure enantiomers and/or diastereoisomers.

Preferred compounds of the invention are optically active isomers. Thus, for example, preferred compounds of formula (I) containing only one chiral centre include an R enantiomer in substantially pure form, an S enantiomer in substantially pure form and enantiomeric mixtures which contain an excess of the R enantiomer or an excess of the S enantiomer.

The compounds of formula (1) may be prepared by conventional routes, for example those set out in any of Schemes 1 to 4 shown below.

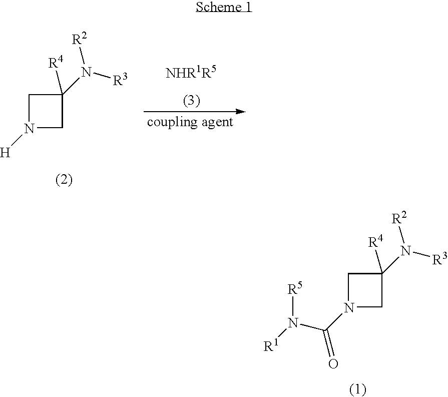

Compounds of formula (1) in which J is —NR⁵— may be prepared, as shown in Scheme 1, from amines of formula (2) and amines of formula 3 together with a carbonyl coupling reagent such as carbonyldiimidazole, phosgene or triphosgene, utilising standard methods such as reaction in solvent such as tetrahydrofuran, acetonitrile, dichloromethane or toluene at a range of temperatures from ambient to reflux temperature. In a preferred embodiment, the compound of formula (3) is a primary amine NH₂R¹. Compounds of formula (3) are either commercially available or may be prepared by standard published methods familiar to those skilled in the art, and compounds of formula (2), which are substituted 3-aminocyclic amine derivatives, may be prepared by standard published methods familiar to those skilled in the art.

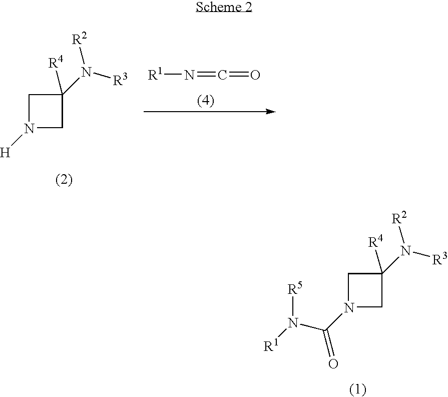

Compounds of formula (1) in which J is —NH— may be prepared, as shown in Scheme 2, from amines of formula (2) and isocyanates of formula (4), utilising standard methods such as reaction in solvent such as tetrahydrofuran, acetonitrile, dichloromethane or toluene at a range of temperatures from ambient to reflux temperature. Compounds of formula (4) are either commercially available or may be prepared by standard published methods familiar to those skilled in the art, and compounds of formula (2), which are substituted 3-aminocyclic amine derivatives, may be prepared by standard published methods familiar to those skilled in the art.

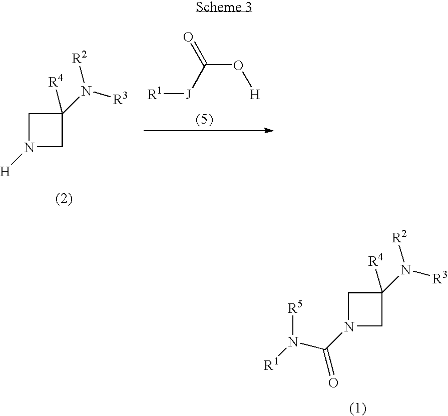

Compounds of formula (1) in which J is a bond may be prepared, as shown in Scheme 3, from heterocyclic amines of formula (2) and carboxylic acids of formula (5) by standard amide coupling conditions, for example in the presence of coupling agents such as EDC/HOBT, DCC or EEDQ, in the presence of a suitable solvent, such as tetrahydrofuran, acetonitrile, dichloromethane or toluene. Carboxylic acids of formula (5) are either commercially available or may be prepared by standard published methods familiar to those skilled in the art, and compounds of formula (2), which are substituted 3-aminocyclic amine derivatives, may be prepared by standard methods familiar to those skilled in the art

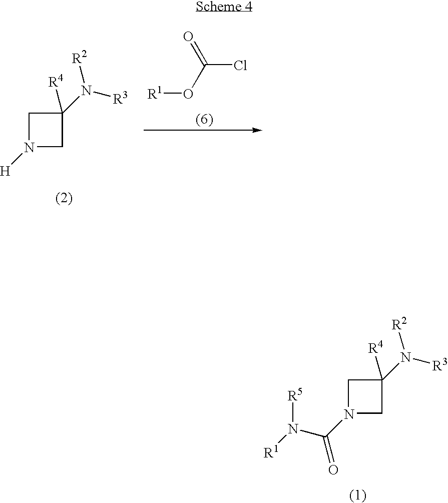

Compounds of formula (1) in which J is —O— may be prepared, as shown in Scheme 4, from substituted secondary amines of formula (2) and a chloroformate of formula (6), utilising standard amide coupling conditions, for example in the presence of a base such as triethylamine, in the presence of a suitable solvent, such as acetonitrile or dichloromethane. Chloroformates of formula (6) are either commercially available or may be prepared by standard published methods familiar to those skilled in the art, and compounds of formula (2), which are substituted 3-aminocyclic amine derivatives, may be prepared by standard methods as outlined above.

The compounds of the invention are found to be inhibitors of sensory neurone specific sodium channels. The compounds of the invention are therefore therapeutically useful. The present invention also provides a compound of formula (I) or a pharmaceutically acceptable salt thereof, as defined above for use in a method of treating the human or animal body. Such compounds are believed to be novel and the present invention also provides for these compounds.

Also provided is a pharmaceutical composition comprising a compound of the formula (1), as defined above, or a pharmaceutically acceptable salt thereof, and a pharmaceutically acceptable carrier or diluent. Said pharmaceutical composition typically contains up to 85 wt % of a compound of the invention. More typically, it contains up to 50 wt % of a compound of the invention. Preferred pharmaceutical compositions are sterile and pyrogen free. Further, the pharmaceutical compositions provided by the invention typically contain a compound of the invention which is a substantially pure optical isomer.

The compounds of the invention may be administered in a variety of dosage forms. Thus, they can be administered orally, for example as tablets, troches, lozenges, aqueous or oily suspensions, dispersible powders or granules. Preferred pharmaceutical compositions of the invention are compositions suitable for oral administration, for example tablets and capsules.

Compositions suitable for oral administration may, if required, contain a colouring or flavoring agent. Typically, a said capsule or tablet comprises from 5 to 500 mg, preferably 10 to 500 mg, more preferably 15 to 100 mg, of a compound of formula (I) or a pharmaceutically acceptable salt thereof.

The compounds of the invention may also be administered parenterally, whether subcutaneously, intravenously, intramuscularly, intrasternally, transdermally or by infusion techniques. The compounds may also be administered as suppositories.

A compound of the invention is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

The compounds of the present invention are therapeutically useful in the treatment or prophylaxis of conditions involving sodium ion flux through a sensory neurone specific (SNS) channel of a sensory neurone. Said condition may be one of hypersensitivity for example resulting from a concentration of SNS channels at the site of nerve injury or in axons following nerve injury, or may be sensitisation of the neurone for example at sites of inflammation as a result of inflammatory mediators.

Said compounds of the invention are therefore most preferred for their use in the treatment or prophylaxis of any condition involving hypersensitivity or sensitisation of a sensory neurone specific (SNS) channel of a sensory neurone.

Accordingly, the present invention also provides the use of a compound of the formula (I), or a pharmaceutically acceptable salt thereof, in the manufacture of a medicament for use in the treatment or prophylaxis of a condition involving sodium ion flux through a sensory neurone specific (SNS) channel of a sensory neurone, more specifically hypersensitivity of a sensory neurone or sensitisation of a sensory neurone specific (SNS) channel of a sensory neurone. Also provided is a method of treating a patient suffering from or susceptible to a condition involving sodium ion flux through a sensory neurone specific (SNS) channel of a sensory neurone, more specifically hypersensitivity of a sensory neurone or sensitisation of a sensory neurone specific (SNS) channel of a sensory neurone, which method comprises administering to said patient an effective amount of a compound of formula (I), or a pharmaceutically acceptable salt thereof.

The term treatment in this context is deemed to cover any effect from a cure of said condition to alleviation of any or all of the symptoms. The compounds of the invention may, where appropriate, be used prophylactically to reduce the incidence or severity of said conditions.

Specific conditions in which SNS channels are present and believed to be involved include pain, for example chronic and acute pain, hypersensitivity disorders such as bladder dysfunction and bowel disorders which may or may not also have associated pain, and demyelinating diseases.

SNS sodium channels are known to mediate pain transmission. Typically, the compounds of the invention are therefore used as analgesic agents. SNS specific sodium channels have been identified as being particularly important in the transmission of pain signals. The compounds of the invention are accordingly particularly effective in alleviating pain. Typically, therefore, said medicament is for use in alleviating pain and said patient is suffering from or susceptible to pain. The compounds of the invention are effective in alleviating both chronic and acute pain.

Acute pain is generally understood to be a constellation of unpleasant sensory, perceptual and emotional experiences of certain associate autonomic (reflex) responses, and of psychological and behavioural reactions provoked by injury or disease. A discussion of acute pain can be found at Halpern (1984) Advances in Pain Research and Therapy, Vol. 7, p. 147. Tissue injury provokes a series of noxious stimuli which are transduced by nociceptors to impulses transmitted to the spinal cord and then to the upper part of the nervous system. Examples of acute pains which can be alleviated with the compounds of the invention include musculoskeletal pain, for example joint pain, lower back pain and neck pain, dental pain, post-operative pain, obstetric pain, for example labour pain, acute headache, neuralgia, myalgia, and visceral pain.

Chronic pain is generally understood to be pain that persists beyond the usual course of an acute disease or beyond a reasonable time for an injury to heal. A discussion of chronic pain can be found in the Halpern reference given above. Chronic pain is sometimes a result of persistent dysfunction of the nociceptive pain system. Examples of chronic pains which can be alleviated with the compounds of the invention include trigeminal neuralgia, post-herpetic neuralgia (a form of chronic pain accompanied by skin changes in a dermatomal distribution following damage by acute Herpes Zoster disease), diabetic neuropathy, causalgia, “phantom limb” pain, pain associated with osteoarthritis, pain associated with rheumatoid arthritis, pain associated with cancer, pain associated with HIV, neuropathic pain, migraine and other conditions associated with chronic cephalic pain, primary and secondary hyperalgesia, inflammatory pain, nociceptive pain, tabes dorsalis, spinal cord injury pain, central pain, post-herpetic pain, noncardiac chest pain, irritable bowel syndrome and pain associated with bowel disorders and dyspepsia.

Some of the chronic pains set out above, for example, trigeminal neuralgia, diabetic neuropathic pain, causalgia, phantom limb pain and central post-stroke pain, have also been classified as neurogenic pain. One non-limiting definition of neurogenic pain is pain caused by dysfunction of the peripheral or central nervous system in the absence of nociceptor stimulation by trauma or disease. The compounds of the invention can, of course, be used to alleviate or reduce the incidence of neurogenic pain

Examples of bowel disorders which can be treated or prevented with the compounds of the invention include inflammatory bowel syndrome and inflammatory bowel disease, for example Crohn's disease and ulcerative colitis.

Examples of bladder dysfunctions which can be treated or prevented with the compounds of the invention include bladder hyper reflexia and bladder inflammation, for example interstitial cystitis, overactive (or unstable) bladder (OAB), more specifically urinary incontinence, urgency, frequency, urge incontinence and nocturia. The compounds of the invention can also be used to alleviate pain associated with bladder hyper reflexia or bladder inflammation.

Examples of demyelinating diseases which can be treated or prevented with the compounds of the invention are those in which SNS channels are known to be expressed by the demyelinated neurones and which may or may not also have associated pain. A specific example of such a demyelinating disease is multiple sclerosis. The compounds of the invention can also be used to alleviate pain associated with demyelinating diseases such as multiple sclerosis.

The compounds of the invention have additional properties as they are capable of inhibiting voltage dependent sodium channels. They can therefore be used, for example, to protect cells against damage or disorders which results from overstimulation of sodium channels.

The compounds of the invention are useful in the treatment and prevention of peripheral and central nervous system disorders. They can therefore additionally be used in the treatment or prevention of an affective disorder, an anxiety disorder, a behavioural disorder, a cardiovascular disorder, a central or peripheral nervous system degenerative disorder, a central nervous system injury, a cerebral ischaemia, a chemical injury or substance abuse disorder, a cognitive disorder, an eating disorder, an eye disease, Parkinson's disease or a seizure disorder.

Examples of affective disorders which can be treated or prevented with the compounds of the invention include mood disorders, bipolar disorders (both Type 1 and Type II) such as seasonal affective disorder, depression, manic depression, atypical depression and monodepressive disease, schizophrenia, psychotic disorders, mania and paranoia.

Examples of anxiety disorders which can be treated or prevented with the compounds of the invention include generalised anxiety disorder (GAD), panic disorder, panic disorder with agoraphobia, simple (specific) phobias (e.g. arachnophobia, performance anxiety such as public speaking), social phobias, post-traumatic stress disorder, anxiety associated with depression, and obsessive compulsive disorder (OCD).

Examples of behavioural disorders which can be treated or prevented with the compounds of the invention include behavioural and psychological signs and symptoms of dementia, age-related behavioural disorders, pervasive development disorders such as autism, Asperger's Syndrome, Retts syndrome and disintegrative disorder, attention deficit disorder, aggressivity, impulse control disorders and personality disorder.

Examples of cardiovascular disorders which can be treated or prevented with the compounds of the invention include cardiac arrthymia, atherosclerosis, cardiac arrest, thrombosis, complications arising from coronary artery bypass surgery, myocardial infarction, reperfusion injury, intermittant claudication, ischaemic retinopathy, angina, pre-eclampsia, hypertension, congestive cardiac failure, restenosis following angioplasty, sepsis and septic shock.

Examples of central and peripheral nervous system degenerative disorders which can be treated or prevented with the compounds of the invention include corticobasal degeneration, disseminated sclerosis, Freidrich's ataxia, motorneurone diseases such as amyotrophic lateral sclerosis and progressive bulbar atrophy, multiple system atrophy, myelopathy, radiculopathy, peripheral neuropathies such as diabetic neuropathy, tabes dorsalis, drug-induced neuropathy and vitamin deficiency, systemic lupus erythamatosis, granulomatous disease, olivo-ponto-cerebellar atrophy, progressive pallidal atrophy, progressive supranuclear palsy and spasticity.

Examples of central nervous system injuries which can be treated with the compounds of the invention include traumatic brain injury, neurosurgery (surgical trauma), neuroprotection for head injuries, raised intracranial pressure, cerebral oedema, hydrocephalus and spinal cord injury.

Examples of cerebral ischaemias which can be treated or prevented with the compounds of the invention include transient ischaemic attack, stroke, for example thrombotic stroke, ischaemic stroke, embolic stroke, haemorrhagic stroke or lacunar stroke, subarachnoid haemorrhage, cerebral vasospasm, peri-natal asphyxia, drowning, cardiac arrest and subdural haematoma.

Examples of chemical injuries and substance abuse disorders which can be treated or prevented with the compounds of the invention include drug dependence, for example opiate dependence, benzodiazepine addition, amphetamine addiction and cocaine addiction, alcohol dependence, methanol toxicity, carbon monoxide poisoning and butane inhalation.

Examples of cognitive disorders which can be treated or prevented with the compounds of the invention include dementia, Alzheimer Disease, Frontotemporal dementia, multi-infarct dementia, AIDS dementia, dementia associated with Huntingtons Disease, Lewy body Dementia, Senile dementia, age-related memory impairment, cognitive impairment associated with dementia, Korsakoff syndrome and dementia pugilans.

Examples of eating disorders which can be treated or prevented with the compounds of the invention include anorexia nervosa, bulimia, Prader-Willi syndrome and obesity.

Examples of eye diseases which can be treated or prevented with the compounds of the invention include drug-induced optic neuritis, cataract, diabetic neuropathy, ischaemic retinopathy, retinal haemorrhage, retinitis pigmentosa, acute glaucoma, in particular acute normal tension glaucoma, chronic glaucoma, in particular chronic normal tension glaucoma, macular degeneration, retinal artery occlusion and retinitis.

Examples of Parkinson's diseases which can be treated or prevented with the compounds of the invention include drug-induced Parkinsonism, post-encephalitic Parkinsonism, Parkinsonism induced by poisoning (for example MPTP, manganese or carbon monoxide poisoning), Dopa-responsive dystonia-Parkinsonism, posttraumatic Parkinson's disease (punch-drunk syndrome), Parkinson's with on-off syndrome, Parkinson's with freezing (end of dose deterioration) and Parkinson's with prominent dyskinesias.

Examples of seizure disorders which can be treated or prevented with the compounds of the invention include epilepsy and post-traumatic epilepsy, partial epilepsy (simple partial seizures, complex partial seizures, and partial seizures secondarily generalised seizures), generalised seizures, including generalised tonicclonic seizures (grand mal), absence seizures (petit mal), myoclonic seizures, atonic seizures, clonic seizures, and tonic seizures, Lennox Gastaut, West Syndome (infantile spasms), multiresistant seizures and seizure prophylaxis (antiepileptogenic).

The compounds of the present invention are also useful in the treatment and prevention of tinnitus.

A therapeutically effective amount of a compound of the invention is administered to a patient. A typical dose is from about 0.001 to 50 mg per kg of body weight, for example 0.01 to 10 mg, according to the activity of the specific compound, the age, weight and conditions of the subject to be treated, the type and severity of the disease and the frequency and route of administration. Preferably, daily dosage levels are from 5 mg to 2 g.

The following Examples including those listed in the Table illustrate the invention. They do not, however, limit the invention in any way. In this regard, it is important to understand that the particular assays used in the Examples section are designed only to provide an indication of activity in inhibiting SNS specific sodium channels. A negative result in any one particular assay is not determinative.

### EXAMPLES

### Example 1

### 3-(1-Methyl-2-o-tolyl-ethylamino)-azetidine-1-carboxylic acid [1-(4-trifluoromethyl-phenyl)-ethyl]-amide (METHOD A)

### i) {1-[1-(4-Trifluoromethyl-phenyl)-ethylcarbamoyl]-azetidin-3-yl}-carbamic acid tent-butyl ester

To a solution of 1-[4-(trifluoromethyl)phenyl]ethylamine (1.0 g, 5.2 mmol) in dichloromethane (20 ml) at 30° C. was added a suspension of 1,1′-carbonyl diimidazole (0.86 g, 5.2 mmol) in dichloromethane (7 ml) and the reaction was stirred at 30° C. for 1 hour. A suspension of azetidine-3-yl-carbamic acid tert-butyl ester (0.89 g, 5.2 mmol) in dichloromethane (5 ml) was added and the reaction stirred at 30° for 5 hours. The reaction was cooled to room temperature (r.t.) and the product obtained by filtration as a white solid. Yield 1.35 g (67%): HPLC retention time, 3.56 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 388 (M+H).

### ii) 3-Amino-azetidine-1-carboxylic acid [1-(4-trifluoromethyl-phenyl)-ethyl]-amide

A solution of the {1-[1-(4-trifluoromethyl-phenyl)-ethylcarbamoyl]-azetidin-3-yl}-carbamic acid tert-butyl ester (1.35 g, 3.5 mmol) in trifluoroacetic acid (8 ml) was stirred at r.t. for 1 hour. The reaction was cooled, diluted with dichloromethane (50 ml) and basified using 50% ammonia. The organic phase was washed (brine), dried and evaporated in vacuo to afford the title compound as a colourless oil. Yield 1.0 g, (99%): HPLC retention time, 2.81 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) Ink 288 (M+H).

### iii) 3-(1-Methyl-2-o-tolyl-ethylamino)-azetidine-1-carboxylic acid [1-(4-trifluoromethyl-phenyl)-ethyl]-amide

A solution of 3-amino-azetidine-1-carboxylic acid [1-(4-trifluoromethyl-phenyl)-ethyl]-amide (97 mg, 0.33 mmol), 2-methylphenylacetone (50 mg, 0.33 mmol) and sodium cyanoborohydride (64 mg, 1.0 mmol) in 1% acetic acid/methanol (10 ml) was heated to reflux for 18 hours. The reaction was cooled to r.t. and basified using ammonia. The mixture was partitioned between dichloromethane and water and the organics collected, dried and evaporated in vacuo. The residue was purified via flash chromatography eluting with EtOAc/methanol (20:1) to afford the title compound as a white solid. Yield 27 mg (20%): HPLC retention time, 3.86 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 420 (M+H).

NMR data for some of the compounds synthesised according to Method A as described for Example 1 using the appropriately substituted starting materials are shown below. Other compounds prepared by this method are listed in the TABLE.

| Example | |
|---|---|
| number | NMR |
| 2 | ¹H NMR (CDCl₃) δ 1.35-1.40 (d, J = 6.09, 3H), 3.45-3.60 (m, 2H), 3.70-3.80 (m, 2H), |
| | 3.90-3.95 (m, 1H), 4.05-4.15 (m, 1H), 4.45-4.52 (d, J = 7.83, 1H), 6.15-6.20 (d, J = 7.83, |
| | 1H), 6.95-7.10 (m, 6H), 7.15-7.30 (m, 7H). |
| 3 | ¹H NMR (CDCl₃) δ 1.49-1.55 (d, J = 7.04, 3H), 2.70-2.80 (m, 2H), 3.10-3.20 (m, 2H), |
| | 3.60-3.82 (m, 4H), 4.15-4.20 (m, 2H), 4.35-4.40 (m, 1H), 5.0-5.10 (m, 1H), |
| | 7.15-7.25 (m, 4H), 7.40-7.50 (m, 2H), 7.55-7.65 (m, 2H). |
| 4 | ¹H NMR (CDCl₃) δ 7.60 (d, 2H), 7.46 (d, 2H), 7.11 (m, 4H), 5.03 (qn, 1H), 4.50 (br, |
| | 1H), 4.18 (dt, 2H), 3.88 (m, br, 1H), 3.79 (br, 2H), 2.94 (m, br, 4H), 2.68 (br, 1H), |
| | 2.06 (br, 1H), 1.68 (br, 1H), 1.50 (d, 3H). |
| 5 | ¹H NMR (CDCl₃) δ 7.61 (d, 2H), 7.45 (d, 2H), 7.02 (m, 1H), 6.82 (m, 2H), 5.04 (qn, |
| | 1H), 4.32 (d, 1H), 4.19 (t, 2H), 3.85 (m, 1H), 3.69 (m, 2H), 2.89 (m, br, 4H), 2.55 (m, |
| | br, 1H), 2.00 (br, 1H), 1.61 (m, br, 2H), 1.50 (d, 3H). |
| 6 | ¹H NMR (CDCl₃) δ 7.27 (m, 4H), 6.90 (m, 4H), 5.90 (s, 1H), 4.22 (m, 3H), 3.94 (t, |
| | 2H), 3.84 (m, 1H), 3.76 (m, 2H), 3.02 (m, br, 1H), 2.82 (m, 1H), 2.40 (m, 1H), |
| | 1.79 (m, 3H), 1.50 (m, 2H), 0.98 (t, 3H). |
| 7 | ¹H NMR (CDCl₃) δ 2.06-2.22 (m, 2H), 2.26 (s, 3H), 2.40-2.44 (m, 1H), 2.87-2.97 (m, |
| | 1H), 3.09-3.54 (m, 1H), 3.94-4.01 (m, 2H), 4.15-4.30 (m, 3H), 4.70-4.80 (s, 1H), |
| | 6.90-7.05 (m, 4H), 7.14-7.30 9 (m, 4H), 7.45-7.55 (m, 2H), 7.60-7.65 (m, 1H), |
| | 9.34-9.44 (s, 1H), 9.44-9.61 (s, 1H). DMSO |
| 8 | ¹H NMR (CDCl₃) δ; 7.33 (d, 2H), 7.03-6.89 (m, 7H), 6.84-6.77 (m, 2H), 5.93 (s, 1H), |
| | 4.27 (t, 2H), 3.91-3.85 (m, 1H), 3.80-3.75 (m, 2H), 3.02-2.77 (m, 4H), 2.59-2.51 (m, |
| | 1H), 2.02-1.95 (m, br, 1H), 1.66-1.56 (m, 1H), 1.51 (s, br, 1H). |
| 9 | ¹H NMR (CDCl₃) δ; 7.33 (d, 2H), 7.13-7.05 (m, 4H), 7.02-6.90 (m, 6H), 5.93 (s, 1H), |
| | 4.28 (dt, 2H), 3.93-3.86 (m, 1H), 3.80-3.75 (m, 2H), 3.02-2.77 (m, 4H), 2.64-2.58 (m, |
| | 1H), 2.04-1.97 (m, br, 1H), 1.68-1.58 (m, 1H), 1.53 (s, br, 1H). |
| 10 | ¹H NMR (CDCl₃) δ; 7.37 (d, 2H), 7.29 (d, 1H), 7.28-7.24 (m, 2H), 6.97-6.90 (m, 6H), |
| | 5.97 (s, 1H), 4.28 (dt, 2H), 4.22 (t, 1H), 3.91-3.85 (m, 1H), 3.82-3.76 (m, 2H), |
| | 3.07-2.99 (m, 1H), 2.87-2.79 (m, 1H), 2.46-2.38 (m, 1H), 1.88-1.79 (m, 1H), 1.59 (s, br, |
| | 1H). |

### Example 22

### 3-(5-Fluoro-indan-1-ylamino)-azetidine-1-carboxylic acid (4-trifluoromethyl-phenyl)-amide (METHOD B)

### i) [1-(4-Trifluoromethyl-phenylcarbamoyl)-azetidin-3-yl]-carbamic acid tert-butyl ester

To a solution of azetidine-3-ylcarbamic acid tert-butyl ester (2.91 g, 16.9 mmol) in dichloromethane (175 mL) was added 4-trifluoromethylphenylisocyanate (3.16 g, 16.9 mmol) and the reaction mixture was stirred for 20 h at room temperature. The reaction mixture was evaporated in vacuo to form the title compound as a white solid. Yield 6 g (99%). HPLC retention time, 3.72 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 360 (M+H).

### ii) 3-Amino-azetidine-1-carboxylic acid (4-trifluoromethyl-phenyl)-amide

The title compound was prepared according to the method described in Example 1 step ii) using [1-(4-trifluoromethyl-phenylcarbamoyl)-azetidin-3-yl]-carbamic acid tert-butyl ester (6.0 g, 16.7 mmol) to afford the title compound as a white solid. Yield 3.5 g (81%): HPLC retention time, 2.93 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 260 (M+H).

### iii) 3-(5-Fluoro-indan-1-ylamino)-azetidine-1-carboxylic acid (4-trifluoromethyl-phenyl)-amide

The title compound was prepared according to the method described in Example 1 step iii) using 3-amino-azetidine-1-carboxylic acid (4-trifluoromethyl-phenyl)-amide (100 mg, 0.4 mmol) and 5-fluoroindanone to afford the title compound as a pale clear gum. Yield 25 mg (16%): HPLC retention time, 3.78 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 394 (M+H).

NMR data for some of the compounds synthesised according to Method B as described for Example 22 using the appropriate starting materials are shown below. Other compounds prepared by this method are listed in the TABLE.

| Example | |
|---|---|
| number | NMR |
| 23 | ¹H NMR (CDCl₃) δ; 0.2-0.35 (m, 2H), 0.42-0.55 (m, 1H), 0.66-0.75 (m, 1H), |
| | 1.05-1.15 (m, 1H), 2.75-2.82 (d, J = 9.09, 1H), 3.60-3.72 (m, 2H), 3.75-3.85 (m, 1H), |
| | 4.50-4.25 (m, 2H), 6.10 (s, 1H), 7.00-7.10 (m, 2H), 7.30-7.35 (m, 2H), 7.50-7.55 (m, 4H). |
| 24 | ¹H NMR (CDCl₃) δ; 1.70-1.82 (m, 1H), 2.30-2.40 (m, 1H), 2.70-2.85 (m, 1H), |
| | 2.90-3.10 (m, 1H), 3.70-3.85 (m, 3H), 4.10-4.35 (m, 3H), 6.60 (s, 1H), 6.82-7.00 (m, 2H), |
| | 7.20-7.40 (m, 3H), 7.60-7.75 (m, 2H). |
| 25 | ¹H NMR (CDCl₃) δ; 1.05-1.10 (d, J = 7.07, 3H), 2.45-2.72 (m, 2H), 2.90-3.00 (m, 1H), |
| | 3.55-3.60 (m, 1H), 3.80-3.90 (m, 2H), 4.02-4.4.08 (d, J = 6.06, (1H), 4.16-4.30 (m, |
| | 2H), 6.15 (s, 1H), 6.95-7.10 (m, 4H), 7.05-7.12 (m, 1H), 7.20-7.40 (m, 8H). |
| 26 | ¹H NMR (CDCl₃) δ; 1.35-1.40 (d, J = 6.82, 3H), 2.60-2.2.70 (m, 1H), |
| | 3.0-3.40 (m. 1H), 3.80-3.60 (m, 3H), 4.10-4.35 (m, 3H), 6.60 (s, 1H), 6.92-7.0 (m, 4H), |
| | 7.50-7.20 (m, 1H), 7.20-7.45 (m, 8H) |

### Example 37

### 3-[(5-Fluoro-indan-1-yl)-methyl-amino]-azetidine-1-carboxylic acid [4-(4-fluoro-phenoxy)-phenyl]-amide (METHOD C)

To a solution of 3-(5-fluoro-indan-1-ylamino)-azetidine-1-carboxylic acid [4-(4-fluoro-phenoxy)-phenyl]-amide (81 mg, 0.19 mmol) in dry methanol (4 ml) was added formaldehyde (220 μl, 37% wt solution/water) followed by sodium cyanoborohydride (46 mg, 0.73 mmol) and the reaction stirred at room temperature (r.t.) for 20 hours. The reaction mixture was evaporated in vacuo and the residue partitioned between dichloromethane (20 ml) and water (5 ml). The organics were collected, dried and evaporated in vacuo. The residue was purified by flash chromatography eluting with EtOAc to afford the title compound as a white solid. Yield 45 mg (54%): HPLC retention time, 4.18 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 450 (M+H).

Other compounds prepared by Method C as described for Example 37 using the appropriate starting materials are listed in the TABLE.

### Example 41

### 3-(5-Fluoro-indan-1-ylamino)-3-methyl-azetidine-1-carboxylic acid [4-(4-fluoro-phenoxy)-phenyl]-amide (METHOD D)

### i) 1-Benzhydryl-3-methyl-azetidin-3-ol hydrochloride salt

Aminodiphenylmethane (16.2 ml, 93.9 mmol) was added dropwise to a solution of 2-chloromethyl-2-methyloxirane (10 g, 94 mmol), in methanol (40 ml) and the reaction stirred at r.t. for 72 hours followed by reflux for 20 hours. The reaction was cooled to r.t., evaporated in vacuo and suspended in acetone (50 ml). The solid material was collected by filtration to afford the title compound as a white solid. Yield 20.4 g, (75%): HPLC retention time, 3.56 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 254 (M+H).

### ii) Methanesulfonic acid 1-benzhydryl-3-methyl-azetidin-3-yl ester

Triethylamine (23 ml, 163.7 mmol) was added dropwise to a suspension of the 1-benzhydryl-3-methyl-azetidin-3-ol hydrochloride salt (20.3 g, 70 mmol) in dichloromethane (160 ml) at 0° C., followed by dropwise addition of a solution of methanesulfonyl chloride (7.3 ml, 94 mmol in 15 ml dichloromethane) such that the temperature did not exceed 5° C. The reaction was stirred for 20 hours at r.t. followed by quenching with the addition of water (50 ml). The organics were collected, dried and evaporated in vacuo. The residue was purified by flash chromatography eluting with EtOAc/Hexanes (1:5) to afford the title compound as a white solid. Yield 5.56 g (24%): HPLC retention time, 4.11 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 332 (M+H).

### iii) 1-Benzhydryl-3-methyl-aminoazetidine

To a solution of ammonia (100 ml, 7N in methanol) was added methanesulfonic acid 1-benzhydryl-3-methyl-azetidin-3-yl ester (5.2 g, 15.8 mmol) and the reaction stirred at r.t. for 20 hours. The solution was evaporated in vacuo and the residue was purified by flash chromatography eluting with Methanol/DCM (1:10) to afford the title compound as a white foam. Yield 3.9 g (98%): HPLC retention time, 3.40 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 253 (M+H).

### iv) (1-Benzhydryl-3-methyl-azetidin-3-yl)-carbamic acid tert-butyl ester

To a solution of the 1-benzhydryl-3-methyl-aminoazetidine (3.7 g, 14.5 mmol) in dichloromethane (40 ml) was added a solution of di-tert-butyl dicarbonate (Aldrich, 19, 913-3) (3.2 g, 14.6 mmol) in dichloromethane (30 ml) followed by triethylamine (2.1 ml, 14.9 mmol) and the reaction was stirred at r.t. for 18 hours. The reaction mixture was diluted with dichloromethane (100 ml) followed by 10% sodium bicarbonate (30 ml) and the organics were collected, dried and evaporated in vacuo to afford the title compound as a white foam. Yield 4.5 g (89%): HPLC retention time, 4.47 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 353 (M+H).

### v) (3-Methyl-azetidin-3-yl)-carbamic acid tert-butyl ester

To a solution of the (1-benzhydryl-3-methyl-azetidin-3-yl)-carbamic acid tert-butyl ester (4.3 g, 12.2 mmol) in diethyl ether (50 ml) was added a solution of 2M HCl in diethyl ether (6.1 ml, 12.2 mmol) and the reaction was stirred at r.t. for 30 min. and evaporated in vacuo. The residue was dissolved in ethanol (100 ml), palladium hydroxide added (20 mol %) and the solution stirred under hydrogen (40 psi) for 20 hours. The solution was filtered, evaporated in vacuo ad the residue washed with benzene (2×15 ml) to afford the title compound as a yellow solid.

### vi) 3-(5-Fluoro-indan-1-ylamino)-3-methyl-azetidine-1-carboxylic acid [4-(4-fluoro-phenoxy)-phenyl]-amide

The methods described in Example 1 using 4-fluorophenoxyphenylaniline and 5-fluoroindan-1-one afforded the title compound as a white foam: HPLC retention time, 4.03 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 450 (M+H).

Other compounds prepared by Method D as described for Example 41 using the appropriate starting materials are listed in the TABLE.

### Example 43

### [3-(5-Fluoro-indan-1-ylamino)-azetidin-1-yl]-[4-(4-fluoro-phenoxy)-phenyl]-methanone (METHOD E)

### i) 3-(5-Fluoro-indan-1-ylamino)-azetidine-1-carboxylic acid tert-butyl ester

To a vigorously stirred solution of titanium(IV) isopropoxide (5 ml, 16.6 mmol), was added solid 5-fluoro-1-indanone (1.8 g, 11.6 mmol) followed by tert-butyl-3-aminoazetidine-1-carboxylate (2 g, 11.6 mmol) and the reaction was stirred at r.t. for 5 hours. Ethanol (40 ml) was added followed by sodium cyanoborohydride (1.3 g, 34.8 mmol), and the reaction was stirred for a further 18 hours at r.t. The reaction was quenched by addition to water (50 ml) and partitioned with dichloromethane (200 ml). The thick suspension was filtered through celite and washed with dichloromethane (2×200 ml). The organic extracts were combined, dried and evaporated and the residue purified by flash chromatography eluting with EtOAc/Hexanes (1:3) to afford the title compound as a white solid. Yield 2.1 g (58%): HPLC retention time, 3.82 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 307 (M+H).

### ii) 3-[(5-Fluoro-indan-1-yl)-(2,2,2-trifluoro-acetyl)-amino]-azetidine-1-carboxylic acid tert-butyl ester

To a solution of the 3-(5-fluoro-indan-1-ylamino)-azetidine-1-carboxylic acid tert-butyl ester (2.06 g 6.7 mmol) in dichloromethane (100 ml) was added triethylamine (940 μl 6.7 mmol) and the reaction was cooled to 0° C. Trifluoroacetic anhydride (960 μl 6.7 mmol) was added and the reaction stirred at r.t. for 24 hours. The reaction mixture was poured onto ice-water (100 ml) and partitioned with dichloromethane (100 ml). The organic extracts were combined, dried, evaporated in vacuo and the residue purified by flash chromatography eluting with EtOAc/Hexanes (1:5) to afford the title compound as an orange oil. Yield 2.65 g (98%): HPLC retention time, 4.4 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 403 (M+H).

### iii) N-Azetidin-3-yl-2,2,2-trifluoro-N-(5-fluoro-indan-1-yl)-acetamide

To a solution of 3-[(5-fluoro-indan-1-yl)-(2,2,2-trifluoro-acetyl)-amino]-azetidine-1-carboxylic acid tert-butyl ester (0.27 g, 0.7 mmol) was added trifluoroacetic acid (5 ml) at 0° C. and the reaction was stirred at r.t. for 30 mins. Dichloromethane (40 ml) was added followed by water (10 ml) and the biphasic mixture basified using 35% aqueous ammonia. The organic extracts were collected, washed (brine), dried and evaporated in vacuo to afford the title compound as a clear gum. Yield 0.2 g (94%): HPLC retention time, 3.65 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 303 (M+H).

### iv) [3-(5-Fluoro-indan-1-ylamino)-azetidin-1-yl]-[4-(4-fluoro-phenoxy)-phenyl]methanone

A solution of N-azetidin-3-yl-2,2,2-trifluoro-N-(5-fluoro-indan-1-yl)-acetamide (0.3 g, 0.99 mmol), 4-(4-fluorophenoxy)benzoic acid (184 mg, 1.2 mmol), EDC (0.2 g, 1.2 mmol), HOBT (0.17 g, 1.2 mmol) and triethylamine (180 μl, 1.2 mmol) in dichloromethane (50 ml) was stirred at r.t. for 24 hours. The reaction mixture was diluted with dichloromethane (50 ml), washed with water (30 ml), dried and evaporated in vacuo. The residue was purified by flash chromatography eluting with EtOAc/Hexanes (1:1), then re-dissolved in methanol (5 ml) and water (5 ml) and potassium carbonate (120 mg) was added and the reaction stirred at r.t. for 18 hours. The organic solvent was removed in vacuo and the residue diluted with water (10 ml). Dichloromethane (20 ml) was added and the organic extracts were collected, dried and evaporated in vacuo to afford the title compound as a pale clear gum. Yield 0.17 g (94%): HPLC retention time, 4.0 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 421 (M+H). ¹H NMR (CDCl₃) δ: 7.61-7.65 (dd, 2H), 7.25 (dd, 2H), 7.02-7.12 (m 4H), 6.86-6.97 (m 4H), 4.4-4.5 (br s 2H), 4.2 (t, 1H), 3.86-4.1 (m 3H), 2.96-3.05 (m 1H), 2.76-2.86 (m 1H), 2.4 (m 1H), 1.85 (m 1H),

Other compounds prepared by Method E as described for Example 43 using the appropriate starting materials are listed in the TABLE.

### Example 45

### 143-(Indan-2-ylamino)-azetidin-1-yl]-2-(4-phenoxy-phenyl)-ethanone (METHOD F)

### i) {1-[2-(4-Phenoxy-phenyl)-acetyl]-azetidin-3-yl}-carbamic acid tert-butyl ester

To a suspension of EDC (2.1 g, 10.96 mmol) in dichloromethane (100 ml) was added N,N-diisopropylethylamine (1.42 g, 10.96 mmol) followed by 4-phenoxyphenylacetic acid (2.5 g, 10.96 mmol) and azetidin-3-yl-carbamic acid tert butyl ester (1.8 g, 10.96 mmol) and the resulting solution was stirred at r.t. for 18 hours. The reaction mixture was quenched with water (50 ml) and the organics collected, dried and evaporated in vacuo. The residue was purified by flash chromatography eluting with EtOAc/hexanes (1:1) to afford the title compound as a white solid. Yield 3.2 g (76%): HPLC retention time, 3.8 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 383 (M+H).

### ii) 1-(3-Amino-azetidin-1-yl)-2-(4-phenoxy-phenyl)-ethanone

To a stirred solution of trifluoroacetic acid (8 ml) was added {1-[2-(4-phenoxy-phenyl)-acetyl]-azetidin-3-yl}-carbamic acid tert-butyl ester (3.2 g, 8.3 mmol) and the reaction mixture stirred at r.t. for 45 mins. The reaction mixture was diluted with dichloromethane (100 ml) and water (30 ml) and the biphasic mixture basified using 35% ammonia solution. The organic extracts were collected, dried and evaporated in vacuo to afford the title compound as an orange gum. Yield 2.1 g (89%). HPLC retention time, 2.9 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 283 (M+H).

### iii) 1-[3-(Indan-2-ylamino)-azetidin-1-yl]-2-(4-phenoxy-phenyl)-ethanone

A solution of 2-indanone (0.14 g, 1.1 mmol), 1-(3-amino-azetidin-1-yl)-2-(4-phenoxy-phenyl)-ethanone (0.3 g, 1.1 mmol) and sodium cyanoborohydride (0.1 g, 1.6 mmol) in methanol/acetic acid (1%) was heated to reflux for 6 hours. The reaction mixture was cooled to r.t. and evaporated in vacuo. The residue was purified by flash chromatography eluting with methanol/EtOAc (1:10) to afford the title compound as a white solid. Yield 0.27 g (65%). HPLC retention time, 3.9 min (Solvent: CH₃CN/H₂O/0.05% NH₃, 5-95% gradient H₂O-6 min. Column: Xterra 50×4.60 i.d., C18 reverse phase. Flow rate: 1.5 mL/min.). Mass spectrum (ES+) m/z 399 (M+H).

Other compounds prepared by Method F as described for Example 45 using the appropriate starting materials are listed in the TABLE.

### Biological Screening

Inhibition of Human Na_{V}1.8 stably expressed in SH-SY-5Y cells

A SH-SY-5Y neuroblastoma cell line stably expressing the human Na_{V}1.8 (hNa_{V}1.8) ion channel was constructed. This cell line has been used to develop a medium to high throughput assay for determining the ability of test compounds to inhibit membrane depolarisation mediated via the hNa_{V}1.8 channel.

SH-SY-5Y hNa_{V}1.8 are grown in adherent monolayer culture using 50:50 Ham's F-12/EMEM tissue culture medium supplemented with 15% (v/v) foetal bovine serum; 2 mM L-glutamine, 1% NEAA and 600 μg·ml⁻¹ Geneticin sulphate. Cells are removed from the tissue culture flask using trypsin/EDTA and re-plated into black walled, clear bottom 96-well assay plates at 50,000 cells·well⁻¹ 24 hours prior to assay.

On the day of assay the cell assay plates are washed to remove cell culture medium using a sodium free assay buffer (145 mM tetramethyl ammonium chloride; 2 mM calcium chloride; 0.8 mM magnesium chloride hexahydrate; 10 mM HEPES; 10 mM glucose; 5 mM potassium chloride, pH 7.4). Fluorescent membrane potential dye solution (FLIPR™ membrane potential dye, Molecular Devices Corporation), containing 10 μM of a pyrethroid to prevent channel inactivation and 250 nM tetrodotoxin (TTX) to reduce interference from TTX-sensitive sodium channels present in the cell line. Test compound, initially dissolved in dimethyl sulfoxide but further diluted in sodium free buffer, is added to achieve the final test concentration range of 100 μM-0.05 μM.

Cell plates are incubated for 30 minutes at room temperature to allow equilibration of dye and test compound. Plates are then transferred to a fluorescence plate reader for fluorescence measurement using an excitation wavelength of 530 nm whilst measuring fluorescence emission at 565 nm. Baseline fluorescence levels are first determined before the addition of a sodium containing buffer (220 mM sodium chloride; 2 mM calcium chloride; 0.8 mM magnesium chloride hexahydrate; 10 mM HEPES; 10 mM glucose; 5 mM potassium chloride. pH 7.4) to cause membrane depolarisation in those cells where channel block has not been effected (final sodium concentration=72.5 mM). Membrane depolarisation is registered by an increase in fluorescence emission at 565 nm.

The change in fluorescence seen in each test well upon the addition of sodium containing buffer is calculated relative to the baseline fluorescence for that well. This figure is then used for calculating the IC₅₀ for each test compound.

TABLE
Summary of synthesis methods, characterisation data and biological activity.

| | | | LCMS | (ES+) | |
|---|---|---|---|---|---|
| Example | | | Ret.n | m/z | hNaᵥ1.8 |
| Number | Compound Name | Method | time* | (M + H) | IC₅₀ |
| 1 | 3-(1-Methyl-2-o-tolyl-ethylamino)-azetidine-1-carboxylic | A | 3.86 | 420 | 1.30 |
| | acid [1-(4-trifluoromethyl-phenyl)-ethyl]-amide | | | | |
| 2 | 3-[1-(4-Fluoro-phenyl)-ethylamino]-azetidine-1- | A | 4.00 | 440 | 2.62 |
| | carboxylic acid [bis-(4-fluoro-phenyl)-methyl]-amide | | | | |
| 3 | 3-(Indan-2-ylamino)-azetidine-1-carboxylic acid [1-(4- | A | 3.65 | 404 | 2.98 |
| | trifluoromethyl-phenyl)-ethyl]-amide | | | | |
| 4 | 3-(1,2,3,4-Tetrahydro-naphthalen-2-ylamino)-azetidine-1- | A | 3.78 | 418 | 3.32 |
| | carboxylic acid [1-(4-trifluoromethyl-phenyl)-ethyl]- | | | | |
| | amide | | | | |
| 5 | 3-(6-Fluoro-1,2,3,4-tetrahydro-naphthalen-2-ylamino)- | A | 3.81 | 436 | 1.74 |
| | azetidine-1-carboxylic acid [1-(4-trifluoromethyl-phenyl)- | | | | |
| | ethyl]-amide | | | | |
| 6 | 3-(5-Fluoro-indan-1-ylamino)-azetidine-1-carboxylic acid | A | 3.88 | 398 | 1.20 |
| | (4-butoxy-phenyl)-amide | | | | |
| 7 | 3-(5-Fluoro-indan-1-ylamino)-azetidine-1-carboxylic acid | A | 3.89 | 436 | 0.25 |
| | [4-(4-fluoro-phenoxy)-phenyl]-amide | | | | |
| 8 | 3-(6-Fluoro-1,2,3,4-tetrahydro-naphthalen-2-ylamino)- | A | 3.96 | 450 | 0.31 |
| | azetidine-1-carboxylic acid [4-(4-fluoro-phenoxy)- | | | | |
| | phenyl]-amide | | | | |
| 9 | 3-(1,2,3,4-Tetrahydro-naphthalen-2-ylamino)-azetidine-1- | A | 3.94 | 432 | |
| | carboxylic acid [4-(4-fluoro-phenoxy)-phenyl]-amide | | | | |
| 10 | 3-(5-Fluoro-indan-1-ylamino)-azetidine-1-carboxylic acid | A | 4.10 | 452 | 30.00 |
| | [4-(4-chloro-phenoxy)-phenyl]-amide | | | | |
| 11 | 3-(5-Fluoro-indan-1-ylamino)-azetidine-1-carboxylic acid | A | 3.97 | 454 | 0.46 |
| | [3-fluoro-4-(4-fluoro-phenoxy)-phenyl]-amide | | | | |
| 12 | 3-(5-Hydroxy-indan-1-ylamino)-azetidine-1-carboxylic | A | 3.37 | 434 | 1.35 |
| | acid [4-(4-fluoro-phenoxy)-phenyl]-amide | | | | |
| 13 | 3-(5-Fluoro-indan-1-ylamino)-azetidine-1-carboxylic acid | A | 3.92 | 436 | 0.31 |
| | [4-(3-fluoro-phenoxy)-phenyl]-amide | | | | |
| 14 | 3-(5-Fluoro-indan-1-ylamino)-azetidine-1-carboxylic acid | A | 3.81 | 436 | 0.54 |
| | [4-(2-fluoro-phenoxy)-phenyl]-amide | | | | |
| 15 | 3-(5-Chloro-indan-1-ylamino)-azetidine-1-carboxylic acid | A | 4.13 | 470 | 0.65 |
| | [3-fluoro-4-(4-fluoro-phenoxy)-phenyl]-amide | | | | |
| 16 | 3-(5-Bromo-indan-1-ylamino)-azetidine-1-carboxylic acid | A | 4.19 | 516 | 1.72 |
| | [3-fluoro-4-(4-fluoro-phenoxy)-phenyl]-amide | | | | |
| 17 | 3-(5-Bromo-indan-1-ylamino)-azetidine-1-carboxylic acid | A | 4.16 | 496 | 0.54 |
| | [4-(4-fluoro-phenoxy)-phenyl]-amide | | | | |
| 18 | 3-(5-Chloro-indan-1-ylamino)-azetidine-1-carboxylic acid | A | 4.10 | 452 | 0.62 |
| | [4-(4-fluoro-phenoxy)-phenyl]-amide | | | | |
| 19 | 3-(1,2,3,4-Tetrahydro-naphthalen-1-ylamino)-azetidine-1- | A | 4.07 | 432 | 0.19 |
| | carboxylic acid [4-(4-fluoro-phenoxy)-phenyl]-amide | | | | |
| 20 | 3-(5-Fluoro-indan-1-ylamino)-azetidine-1-carboxylic acid | A | 4.05 | 450 | 0.70 |
| | [4-(4-fluoro-phenoxy)-phenyl]-methyl-amide | | | | |
| 21 | 3-(5-Methoxy-indan-1-ylamino)-azetidine-1-carboxylic | A | 3.78 | 448 | 0.31 |
| | acid [4-(4-fluoro-phenoxy)-phenyl]-amide | | | | |
| 22 | 3-(5-Fluoro-indan-1-ylamino)-azetidine-1-carboxylic acid | B | 3.78 | 394 | 3.11 |
| | (4-trifluoromethyl-phenyl)-amide | | | | |
| 23 | 3-{[Cyclopropyl-(4-fluoro-phenyl)-methyl]-amino}- | B | 3.92 | 408 | 30.00 |
| | azetidine-1-carboxylic acid (4-trifluoromethyl-phenyl)- | | | | |
| | amide | | | | |
| 24 | 3-(5-Fluoro-indan-1-ylamino)-azetidine-1-carboxylic acid | B | 3.79 | 394 | 2.68 |
| | (3-trifluoromethyl-phenyl)-amide | | | | |
| 25 | 3-(2-Methyl-indan-1-ylamino)-azetidine-1-carboxylic | B | 4.09 | 414 | 0.84 |
| | acid (4-phenoxy-phenyl)-amide | | | | |
| 26 | 3-(3-Methyl-indan-1-ylamino)-azetidine-1-carboxylic | B | 4.05 | 414 | 0.24 |
| | acid (4-phenoxy-phenyl)-amide | | | | |
| 27 | 3-(5-Fluoro-indan-1-ylamino)-azetidine-1-carboxylic acid | B | 3.86 | 418 | 0.27 |
| | (4-phenoxy-phenyl)-amide | | | | |
| 28 | 3-(5-Fluoro-indan-1-ylamino)-azetidine-1-carboxylic acid | B | 3.96 | 418 | 0.43 |
| | (3-phenoxy-phenyl)-amide | | | | |
| 29 | 3-[(5-Fluoro-indan-1-yl)-methyl-amino]-azetidine-1- | B | 4.26 | 432 | 0.42 |
| | carboxylic acid (3-phenoxy-phenyl)-amide | | | | |
| 30 | 3-(Indan-1-ylamino)-azetidine-1-carboxylic acid (4- | B | 3.85 | 400 | 0.26 |
| | phenoxy-phenyl)-amide | | | | |
| 31 | 3-(5-Fluoro-indan-1-ylamino)-azetidine-1-carboxylic acid | B | 3.70 | 430 | 7.25 |
| | (4-benzoyl-phenyl)-amide | | | | |
| 32 | 3-(1,2,3,4-Tetrahydro-naphthalen-2-ylamino)-azetidine-1- | B | 3.81 | 390 | 0.45 |
| | carboxylic acid (4-trifluoromethyl-phenyl)-amide | | | | |
| 33 | 3-(5-Hydroxy-indan-1-ylamino)-azetidine-1-carboxylic | B | 3.21 | 392 | 11.51 |
| | acid (4-trifluoromethyl-phenyl)-amide | | | | |
| 34 | 3-(1,2,3,4-Tetrahydro-naphthalen-2-ylamino)-azetidine-1- | B | 3.90 | 414 | 0.10 |
| | carboxylic acid (4-phenoxy-phenyl)-amide | | | | |
| 35 | 3-(6-Fluoro-1,2,3,4-tetrahydro-naphthalen-2-ylamino)- | B | 3.83 | 408 | 1.31 |
| | azetidine-1-carboxylic acid (4-trifluoromethyl-phenyl)- | | | | |
| | amide | | | | |
| 36 | 3-(1,2,3,4-Tetrahydro-naphthalen-1-ylamino)-azetidine-1- | B | 3.97 | 390 | 2.77 |
| | carboxylic acid (4-trifluoromethyl-phenyl)-amide | | | | |
| 37 | 3-[(5-Fluoro-indan-1-yl)-methyl-amino]-azetidine-1- | C | 4.18 | 450 | 0.42 |
| | carboxylic acid [4-(4-fluoro-phenoxy)-phenyl]-amide | | | | |
| 38 | 3-[(5-Fluoro-indan-1-yl)-methyl-amino]-azetidine-1- | C | 4.13 | 432 | 0.56 |
| | carboxylic acid (4-phenoxy-phenyl)-amide | | | | |
| 39 | 3[(5-Fluoro-indan-1-yl)-methyl-amino]-azetidine-1- | C | 4.07 | 408 | 2.02 |
| | carboxylic acid (4-trifluoromethyl-phenyl)-amide | | | | |
| 40 | 3[(5-Fluoro-indan-1-yl)-methyl-amino]-azetidine-1- | C | 4.09 | 408 | 2.30 |
| | carboxylic acid (3-trifluoromethyl-phenyl)-amide | | | | |
| 41 | 3-(5-Fluoro-indan-1-ylamino)-3-methyl-azetidine-1- | D | 4.03 | 450 | 0.56 |
| | carboxylic acid [4-(4-fluoro-phenoxy)-phenyl]-amide | | | | |
| 42 | 3-Methyl-3-(1,2,3,4-tetrahydro-naphthalen-2-ylamino)- | D | 3.85 | 404 | 0.69 |
| | azetidine-1-carboxylic acid (4-trifluoromethyl-phenyl)- | | | | |
| | amide | | | | |
| 43 | [3-(5-Fluoro-indan-1-ylamino)-azetidin-1-yl]-[4-(4- | E | 4.00 | 421 | 0.24 |
| | fluoro-phenoxy)-phenyl]-methanone | | | | |
| 44 | (4-Phenoxy-phenyl)-[3-(1-phenyl-ethylamino)-azetidin-1- | E | 4.03 | 373 | 0.73 |
| | yl]-methanone | | | | |
| 45 | 1-[3-(Indan-2-ylamino)-azetidin-1-yl]-2-(4-phenoxy- | F | 3.86 | 399 | 0.65 |
| | phenyl)-ethanone | | | | |
| 46 | 2-(4-Phenoxy-phenyl)-1-[3-(1-phenyl-ethylamino)- | F | 4.00 | 387 | 1.07 |
| | azetidin-1-yl]-ethanone | | | | |
| 47 | 1-[3-(5-Fluoro-indan-1-ylamino)-azetidin-1-yl]-2-(4- | F | 3.97 | 417 | 1.66 |
| | phenoxy-phenyl)-ethanone | | | | |
| 48 | 1-[3-(5-Fluoro-indan-1-ylamino)-azetidin-1-yl]-2-[4-(4- | F | 3.93 | 435 | 0.69 |
| | fluoro-phenoxy)-phenyl]-ethanone | | | | |
| 49 | 2-[4-(4-Fluoro-phenoxy)-phenyl]-1-[3-(indan-2-ylamino)- | F | 3.91 | 417 | 0.43 |
| | azetidin-1-yl]-ethanone | | | | |
| 50 | 1-[3-(5-Fluoro-indan-2-ylamino)-azetidin-1-yl]-2-[4-(4- | F | 3.87 | 435 | 0.78 |
| | fluoro-phenoxy)-phenyl]-ethanone | | | | |
| 51 | 3-(2-Allyloxy-benzylamino)-azetidine-1-carboxylic acid | B | 4.03 | 444 | 0.34 |
| | (4-benzyloxy-phenyl)-amide | | | | |
| 52 | 3-(2,6-Difluoro-benzylamino)-azetidine-1-carboxylic acid | B | 3.87 | 424 | 0.87 |
| | (4-benzyloxy-phenyl)-amide | | | | |
| 53 | [1-(4-Phenoxy-phenylcarbamoyl)-azetidin-3-ylamino]- | B | 3.88 | 432 | 2.23 |
| | phenyl-acetic acid methyl ester | | | | |
| 54 | 3-[1-(4-Methoxy-phenyl)-ethylamino]-azetidine-1- | B | 3.90 | 418 | 0.42 |
| | carboxylic acid (4-phenoxy-phenyl)-amide | | | | |
| 55 | 3-[(Carbamoyl-phenyl-methyl)-amino]-azetidine-1- | H | 3.42 | 417 | 7.71 |
| | carboxylic acid (4-phenoxy-phenyl)-amide | | | | |
| 56 | 3-[1-(4-Fluoro-phenyl)-ethylamino]-azetidine-1- | B | 3.88 | 382 | 3.28 |
| | carboxylic acid (4-trifluoromethyl-phenyl)-amide | | | | |
| 57 | 3-[1-(2-Chloro-4-fluoro-phenyl)-ethylamino]-azetidine-1- | B | 4.28 | 440 | 1.22 |
| | carboxylic acid (4-phenoxy-phenyl)-amide | | | | |
| 58 | 3-[1-(2-Chloro-4-fluoro-phenyl)-ethylamino]-azetidine-1- | B | 4.28 | 440 | 1.12 |
| | carboxylic acid (3-phenoxy-phenyl)-amide | | | | |
| 59 | 3-{[1-(2-Chloro-4-fluoro-phenyl)-ethyl]-methyl-amino}- | G | 4.18 | 430 | 1.87 |
| | azetidine-1-carboxylic acid (4-trifluoromethyl-phenyl)- | | | | |
| | amide | | | | |
| 60 | 3-{[1-(2-Chloro-4-fluoro-phenyl)-ethyl]-methyl-amino}- | C | 4.35 | 454 | 0.57 |
| | azetidine-1-carboxylic acid (4-phenoxy-phenyl)-amide | | | | |
| 61 | 3-(1-o-Tolyl-ethylamino)-azetidine-1-carboxylic acid (4- | B | 3.93 | 402 | 0.75 |
| | phenoxy-phenyl)-amide | | | | |
| 62 | 3-[Methyl-(1-o-tolyl-ethyl)-amino]-azetidine-1- | C | 4.30 | 416 | 0.72 |
| | carboxylic acid (4-phenoxy-phenyl)-amide | | | | |
| 63 | 3-[1-(2-Chloro-phenyl)-ethylamino]-azetidine-1- | B | 4.06 | 422 | 0.63 |
| | carboxylic acid (4-phenoxy-phenyl)-amide | | | | |
| 64 | 3-(8-Methoxy-3,4-dihydro-1H-isoquinolin-2-yl)- | K | 3.82 | 406 | 30.00 |
| | azetidine-1-carboxylic acid (4-trifluoromethyl-phenyl)- | | | | |
| | amide | | | | |
| 65 | 3-(1-o-Tolyl-ethylamino)-azetidine-1-carboxylic acid (4- | B | 3.09 | 382 | 1.42 |
| | butoxy-phenyl)-amide | | | | |
| 66 | 3-[1-(4-Fluoro-phenyl)-ethylamino]-azetidine-1- | B | 3.84 | 386 | 2.27 |
| | carboxylic acid (4-butoxy-phenyl)-amide | | | | |
| 67 | 3-{[1-(2-Chloro-phenyl)-ethyl]-methyl-amino}-azetidine- | C | 4.28 | 436 | 0.55 |
| | 1-carboxylic acid (4-phenoxy-phenyl)-amide | | | | |
| 68 | 3-(1-o-Tolyl-ethylamino)-azetidine-1-carboxylic acid (3- | A | 4.02 | 382 | 0.83 |
| | butoxy-phenyl)-amide | | | | |
| 69 | 3-[1-(4-Fluoro-phenyl)-ethylamino]-azetidine-1- | A | 3.92 | 386 | 1.32 |
| | carboxylic acid (3-butoxy-phenyl)-amide | | | | |
| 70 | 3-[1-(4-Trifluoromethoxy-phenyl)-ethylamino]-azetidine- | B | 4.17 | 472 | 22.15 |
| | 1-carboxylic acid (4-phenoxy-phenyl)-amide | | | | |
| 71 | 3-[1-(2-Chloro-phenyl)-ethylamino]-azetidine-1- | B | 4.05 | 402 | 0.77 |
| | carboxylic acid (4-butoxy-phenyl)-amide | | | | |
| 72 | 3-[1-(4-Fluoro-phenyl)-ethylamino]-azetidine-1- | B | 3.77 | 398 | 3.21 |
| | carboxylic acid (4-trifluoromethoxy-phenyl)-amide | | | | |
| 73 | 3-[1-(4-Fluoro-phenyl)-ethylamino]-azetidine-1- | B | 3.76 | 382 | 2.56 |
| | carboxylic acid (3-trifluoromethyl-phenyl)-amide | | | | |
| 74 | 3-[1-(2-Chloro-phenyl)-ethylamino]-azetidine-1- | B | 3.96 | 398 | 2.86 |
| | carboxylic acid (3-trifluoromethyl-phenyl)-amide | | | | |
| 75 | 3-(1-o-Tolyl-ethylamino)-azetidine-1-carboxylic acid (3- | B | 3.85 | 378 | 2.26 |
| | trifluoromethyl-phenyl)-amide | | | | |
| 76 | 3-(8-Methoxy-3,4-dihydro-1H-isoquinolin-2-yl)- | K | 3.95 | 410 | 1.41 |
| | azetidine-1-carboxylic acid (4-butoxy-phenyl)-amide | | | | |
| 77 | 3-[1-(4-Phenoxy-phenyl)-ethylamino]-azetidine-1- | B | 4.26 | 480 | 1.09 |
| | carboxylic acid (4-phenoxy-phenyl)-amide | | | | |
| 78 | 3-[1-(4-Phenoxy-phenyl)-ethylamino]-azetidine-1- | B | 4.20 | 456 | 1.30 |
| | carboxylic acid (4-trifluoromethyl-phenyl)-amide | | | | |
| 79 | 3-[1-(4-Fluoro-phenyl)-ethylamino]-azetidine-1- | B | 3.83 | 406 | 0.76 |
| | carboxylic acid (4-phenoxy-phenyl)-amide | | | | |
| 80 | 3-{Methyl-[1-(4-trifluoromethoxy-phenyl)-ethyl]- | C | 4.40 | 486 | 1.22 |
| | amino}-azetidine-1-carboxylic acid (4-phenoxy-phenyl)- | | | | |
| | amide | | | | |
| 81 | 3-{[1-(4-Fluoro-phenyl)-ethyl]-methyl-amino}-azetidine- | G | 4.02 | 414 | 8.37 |
| | 1-carboxylic acid (2-fluoro-5-trifluoromethyl-phenyl)- | | | | |
| | amide | | | | |
| 82 | 3-{[1-(4-Fluoro-phenyl)-ethyl]-methyl-amino}-azetidine- | G | 4.13 | 414 | 5.73 |
| | 1-carboxylic acid (3-fluoro-5-trifluoromethyl-phenyl)- | | | | |
| | amide | | | | |
| 83 | 3-{[1-(4-Fluoro-phenyl)-ethyl]-methyl-amino}-azetidine- | G | 3.99 | 414 | 3.93 |
| | 1-carboxylic acid (4-fluoro-3-trifluoromethyl-phenyl)- | | | | |
| | amide | | | | |
| 84 | 3-{[1-(4-Fluoro-phenyl)-ethyl]-methyl-amino}-azetidine- | G | 3.98 | 414 | 5.80 |
| | 1-carboxylic acid (2-fluoro-3-trifluoromethyl-phenyl)- | | | | |
| | amide | | | | |
| 85 | 3-{[1-(4-Fluoro-phenyl)-ethyl]-methyl-amino}-azetidine- | C | 4.14 | 400 | 1.46 |
| | 1-carboxylic acid (3-butoxy-phenyl)-amide | | | | |
| 86 | 3-{[1-(4-Fluoro-phenyl)-ethyl]-methyl-amino}-azetidine- | C | 4.04 | 400 | 3.53 |
| | 1-carboxylic acid (4-butoxy-phenyl)-amide | | | | |
| 87 | 3-[Methyl-(1-o-tolyl-ethyl)-amino]-azetidine-1- | C | 4.40 | 396 | 1.03 |
| | carboxylic acid (3-butoxy-phenyl)-amide | | | | |
| 88 | 3-[1-(2-Trifluoromethyl-phenyl)-ethylamino]-azetidine-1- | B | 4.18 | 456 | 1.18 |
| | carboxylic acid (4-phenoxy-phenyl)-amide | | | | |
| 89 | 3-[Methyl-(1-o-tolyl-ethyl)-amino]-azetidine-1- | C | 4.30 | 396 | 0.58 |
| | carboxylic acid (4-butoxy-phenyl)-amide | | | | |
| 90 | 3-{Methyl-[1-(4-phenoxy-phenyl)-ethyl]-amino}- | C | 4.44 | 470 | 1.43 |
| | azetidine-1-carboxylic acid (4-trifluoromethyl-phenyl)- | | | | |
| | amide | | | | |
| 91 | 3-{[1-(4-Fluoro-phenyl)-ethyl]-methyl-amino}-azetidine- | C | 4.06 | 420 | 0.69 |
| | 1-carboxylic acid (4-phenoxy-phenyl)-amide | | | | |
| 92 | 3-[[1-(2-Chloro-4-fluoro-phenyl)-ethyl]-(2,2,2-trifluoro- | G | 4.44 | 536 | 15.07 |
| | acetyl)-amino]-azetidine-1-carboxylic acid (4-phenoxy- | | | | |
| | phenyl)-amide | | | | |
| 93 | 3-{Methyl-[1-(2-trifluoromethyl-phenyl)-ethyl]-amino}- | C | 4.50 | 470 | 1.80 |
| | azetidine-1-carboxylic acid (4-phenoxy-phenyl)-amide | | | | |
| 94 | 3-[1-(2-Chloro-phenyl)-ethylamino]-azetidine-1- | A | 4.09 | 440 | 0.54 |
| | carboxylic acid [4-(4-fluoro-phenoxy)-phenyl]-amide | | | | |
| 95 | 3-{[1-(4-Fluoro-phenyl)-ethyl]-methyl-amino}-azetidine- | C | 3.99 | 396 | 4.55 |
| | 1-carboxylic acid (3-trifluoromethyl-phenyl)-amide | | | | |
| 96 | 3-[Methyl-(1-o-tolyl-ethyl)-amino]-azetidine-1- | C | 4.26 | 392 | 3.02 |
| | carboxylic acid (3-trifluoromethyl-phenyl)-amide | | | | |
| 97 | 3-{[1-(2-Chloro-phenyl)-ethyl]-methyl-amino}-azetidine- | C | 4.25 | 412 | 3.64 |
| | 1-carboxylic acid (3-trifluoromethyl-phenyl)-amide | | | | |
| 98 | 3-{[1-(4-Fluoro-phenyl)-ethyl]-methyl-amino}-azetidine- | C | 4.01 | 412 | 3.19 |
| | 1-carboxylic acid (4-trifluoromethoxy-phenyl)-amide | | | | |
| 99 | 3-[Methyl-(1-methyl-1-phenyl-ethyl)-amino]-azetidine-1- | G | 4.36 | 416 | 0.63 |
| | carboxylic acid (4-phenoxy-phenyl)-amide | | | | |
| 100 | 3-{[1-(2-Chloro-4-fluoro-phenyl)-ethyl]-methyl-amino}- | G | 4.46 | 454 | 1.21 |
| | azetidine-1-carboxylic acid (3-phenoxy-phenyl)-amide | | | | |
| 101 | 3-[1-(2,4-Dimethyl-phenyl)-ethylamino]-azetidine-1- | B | 4.13 | 416 | 0.29 |
| | carboxylic acid (4-phenoxy-phenyl)-amide | | | | |
| 102 | 3-[1-(2-Chloro-phenyl)-ethylamino]-azetidine-1- | B | 4.12 | 422 | 0.80 |
| | carboxylic acid (3-phenoxy-phenyl)-amide | | | | |
| 103 | 3-[1-(4-Fluoro-phenyl)-ethylamino]-azetidine-1- | B | 3.90 | 406 | 0.73 |
| | carboxylic acid (3-phenoxy-phenyl)-amide | | | | |
| 104 | 3-(1-o-Tolyl-ethylamino)-azetidine-1-carboxylic acid (4- | B | 3.86 | 378 | 2.54 |
| | trifluoromethyl-phenyl)-amide | | | | |
| 105 | 3-{[1-(2,4-Dimethyl-phenyl)-ethyl]-methyl-amino}- | C | 4.51 | 430 | 0.42 |
| | azetidine-1-carboxylic acid (4-phenoxy-phenyl)-amide | | | | |
| 106 | 3-(1-o-Tolyl-ethylamino)-azetidine-1-carboxylic acid (3- | B | 4.02 | 402 | 0.56 |
| | phenoxy-phenyl)-amide | | | | |
| 107 | 3-[Methyl-(1-o-tolyl-ethyl)-amino]-azetidine-1- | C | 4.39 | 416 | 0.51 |
| | carboxylic acid (3-phenoxy-phenyl)-amide | | | | |
| 108 | 3-(1-o-Tolyl-ethylamino)-azetidine-1-carboxylic acid [4- | A | 3.97 | 420 | 0.27 |
| | (4-fluoro-phenoxy)-phenyl]-amide | | | | |
| 109 | 3-{[1-(4-Fluoro-phenyl)-ethyl]-methyl-amino}-azetidine- | C | 4.12 | 420 | 0.47 |
| | 1-carboxylic acid (3-phenoxy-phenyl)-amide | | | | |
| 110 | 3-[Methyl-(1-o-tolyl-ethyl)-amino]-azetidine-1- | C | 4.23 | 392 | 2.24 |
| | carboxylic acid (4-trifluoromethyl-phenyl)-amide | | | | |
| 111 | 3-[1-(4-Fluoro-2-methyl-phenyl)-ethylamino]-azetidine- | B | 4.01 | 420 | 0.63 |
| | 1-carboxylic acid (4-phenoxy-phenyl)-amide | | | | |
| 112 | 3-[1-(4-Fluoro-2-methyl-phenyl)-ethylamino]-azetidine- | B | 4.04 | 420 | 0.66 |
| | 1-carboxylic acid (3-phenoxy-phenyl)-amide | | | | |
| 113 | 3-[1-(4-Fluoro-2-methyl-phenyl)-ethylamino]-azetidine- | B | 3.89 | 396 | 2.84 |
| | 1-carboxylic acid (3-trifluoromethyl-phenyl)-amide | | | | |
| 114 | 3-[1-(4-Fluoro-2-methyl-phenyl)-ethylamino]-azetidine- | B | 3.91 | 396 | 2.30 |
| | 1-carboxylic acid (4-trifluoromethyl-phenyl)-amide | | | | |
| 115 | 3-[1-(4-Methoxy-phenyl)-ethylamino]-azetidine-1- | B | 3.65 | 394 | 2.43 |
| | carboxylic acid (4-trifluoromethyl-phenyl)-amide | | | | |
| 116 | 3-(5-Fluoro-1,3-dioxo-1,3-dihydro-isoindol-2-yl)- | I | 3.82 | 432 | 30.00 |
| | azetidine-1-carboxylic acid (4-phenoxy-phenyl)-amide | | | | |
| 117 | 3-{[1-(4-Fluoro-2-methyl-phenyl)-ethyl]-methyl-amino}- | C | 4.44 | 434 | 0.89 |
| | azetidine-1-carboxylic acid (4-phenoxy-phenyl)-amide | | | | |
| 118 | 3-(1-m-Tolyl-ethylamino)-azetidine-1-carboxylic acid (4- | B | 3.92 | 402 | 0.43 |
| | phenoxy-phenyl)-amide | | | | |
| 119 | 3-[1-(4-Fluoro-2-methyl-phenyl)-ethylamino]-azetidine- | A | 4.01 | 438 | 0.46 |
| | 1-carboxylic acid [4-(4-fluoro-phenoxy)-phenyl]-amide | | | | |
| 120 | 3-[1-(4-Piperidin-1-yl-phenyl)-ethylamino]-azetidine-1- | B | 4.26 | 471 | 0.59 |
| | carboxylic acid (4-phenoxy-phenyl)-amide | | | | |
| 121 | 3-(1-p-Tolyl-ethylamino)-azetidine-1-carboxylic acid (4- | B | 3.96 | 402 | 0.40 |
| | phenoxy-phenyl)-amide | | | | |
| 122 | 3-(1-p-Tolyl-ethylamino)-azetidine-1-carboxylic acid (4- | B | 3.86 | 378 | 1.70 |
| | trifluoromethyl-phenyl)-amide | | | | |
| 123 | 3-(2,3-Dihydro-benzofuran-3-ylamino)-azetidine-1- | B | 3.63 | 402 | 1.70 |
| | carboxylic acid (4-phenoxy-phenyl)-amide | | | | |
| 124 | 3-[1-(2-Chloro-phenyl)-ethylamino]-azetidine-1- | B | 3.96 | 398 | 2.48 |
| | carboxylic acid (4-trifluoromethyl-phenyl)-amide | | | | |
| 125 | 3-(5-Fluoro-1,3-dioxo-1,3-dihydro-isoindol-2-yl)- | I | 3.76 | 408 | 30.00 |
| | azetidine-1-carboxylic acid (4-trifluoromethyl-phenyl)- | | | | |
| | amide | | | | |
| 126 | 3-[1-(4-Cyano-phenyl)-ethylamino]-azetidine-1- | B | 3.58 | 389 | 23.70 |
| | carboxylic acid (4-trifluoromethyl-phenyl)-amide | | | | |
| 127 | 3-[1-(4-Cyano-phenyl)-ethylamino]-azetidine-1- | B | 3.70 | 431 | 2.11 |
| | carboxylic acid [4-(4-fluoro-phenoxy)-phenyl]-amide | | | | |
| 128 | 3-(2,6-Difluoro-benzylamino)-azetidine-1-carboxylic acid | B | 3.73 | 410 | 0.83 |
| | (4-phenoxy-phenyl)-amide | | | | |
| 129 | 3-(2,6-Difluoro-benzylamino)-azetidine-1-carboxylic acid | A | 3.75 | 428 | 1.49 |
| | [4-(4-fluoro-phenoxy)-phenyl]-amide | | | | |
| 130 | 3-[1-(2,6-Difluoro-phenyl)-ethylamino]-azetidine-1- | B | 3.87 | 424 | 0.75 |
| | carboxylic acid (4-phenoxy-phenyl)-amide | | | | |
| 131 | 3-[1-(2,6-Difluoro-phenyl)-ethylamino]-azetidine-1- | A | 3.88 | 442 | 0.53 |
| | carboxylic acid [4-(4-fluoro-phenoxy)-phenyl]-amide | | | | |
| 132 | 3-(2-Methyl-benzoylamino)-azetidine-1-carboxylic acid | B | 3.58 | 402 | 30.00 |
| | (4-phenoxy-phenyl)-amide | | | | |
| 133 | 3-(5-Chloro-indan-1-ylamino)-azetidine-1-carboxylic acid | A | 3.99 | 452 | 1.55 |
| | [4-(2-fluoro-phenoxy)-phenyl]-amide | | | | |
| 134 | 3-(5-Methoxy-indan-1-ylamino)-azetidine-1-carboxylic | B | 3.82 | 448 | 0.17 |
| | acid [4-(3-fluoro-phenoxy)-pheny]-amide | | | | |
| 135 | 3-(5-Fluoro-indan-1-ylamino)-azetidine-1-carboxylic acid | J | 3.58 | 437 | 2.17 |
| | [6-(4-fluoro-phenoxy)-pyridin-3-yl]-amide | | | | |
| | methanesulfonate salt | | | | |
| 136 | 3-(5-Fluoro-indan-1-ylamino)-azetidine-1-carboxylic acid | Salt | 3.91 | 436 | 0.21 |
| | [4-(4-fluoro-phenoxy)-phenyl]-amide methanesulfonate | | | | |
| | salt | | | | |
| 137 | 3-(5-Fluoro-indan-1-ylamino)-azetidine-1-carboxylic acid | Salt | 3.84 | 418 | 0.27 |
| | (4-phenoxy-phenyl)-amide fumarate salt | | | | |
| 138 | 3-(5-Chloro-indan-1-ylamino)-azetidine-1-carboxylic acid | Salt | 4.07 | 452 | 0.43 |
| | [4-(4-fluoro-phenoxy)-phenyl]-amide methanesulfonate | | | | |
| | salt | | | | |

Notes:
*Solvent: CH₃CN/H₂O/0.05% NH₃, 5-95% gradient H₂O-6 min. Column: Xterra 50 × 4.60 i.d., C18 reverse phase. Flow rate: 1.5 mL/min.
Method G - similar to Method B except urea formation is the final step
Method H - similar to Method B except alkylation and amide formation replaces reductive amination
Method I - as Method B except reaction with phthalic anhydride replaces reductive amination as final step
Method J - as Method E except urea formation using aniline and triphosgene replaces final amide coupling
Method K - from the appropriate aromatic isocyanate and substituted azetidine Salts were typically prepared by evaporation of an equimolar solution of the parent compound and appropriate acid in DCM, followed by trituration with ether.

1. Use of a compound of the formula (I), or a pharmaceutically acceptable salt thereof, in the manufacture of a medicament for use in the treatment or prevention of a condition involving sodium ion flux through a sensory neurone specific channel of a sensory neurone 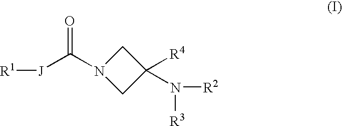
R¹ represents:
(a) -L-A or -L′-A′ wherein L represents a bond or a C₁-C₆ alkyl, C₂-C₆ alkenyl or C₂-C₆ alkynyl moiety, A represents a phenyl, 5- to 10-membered heteroaryl, C₃-C₆ carbocyclyl or 5- to 10-membered heterocyclyl group, L′ represents a C₁-C₆ alkyl, C₂-C₆ alkenyl or C₂-C₆ alkynyl moiety, and A′ represents -Het-A or —X-A wherein Het represents —O—, —S— or —NR¹—, and X represents —CO—, —SO—, —SO₂—, —CO—O—, —CO—S—, —CONR¹—, —O—CO—, —S—CO— or —NR¹—OC—, wherein R¹ represents hydrogen or C₁-C₆ alkyl;
(b) -L-CR(A)(A′) or -L-CR(A)(L-A) wherein R is hydrogen or C₁-C₄ alkyl, A′ is as defined above, each L is the same or different and is as defined above and each A is the same or different and is as defined above;
(c) -L-A-A′ or -L-A-L-A wherein A′ and L are as defined above and each A is the same or different and is as defined above; or
(d) -A-Z-A wherein Z is -Het-L′-, —X-L′-, -L′-Het- or -L′-X—, wherein Het, L′ and X are as defined above and each A is the same or different and is as defined above;
J represents —NR⁵—, —O— or a direct bond wherein R⁵ represents hydrogen, C₁-C₆ alkyl, C₂-C₆ alkenyl or C₂-C₆ alkynyl;
R⁴ is represents hydrogen, C₁-C₆ alkyl, C₂-C₆ alkenyl or C₂-C₆ alkynyl; and either
R² represents -L-A, -L-A′, -L-A-A′, -L-A-L-A, -L-CR(A)(L-A), -L-CR(A)(A′) or -L-CR(A)(L″) wherein L″ is -Het-L′, —X-L′, —CONH₂ or —CO₂H, and wherein A′, Het, X and R are as defined above, each L is the same or different and is as defined above, each A is the same or different and is as defined above and each L′ is the same or different and is as defined above, and
R³ represents hydrogen, C₁-C₆ alkyl, C₂-C₆ alkenyl, C₂-C₆ alkynyl or —(CO)-L′,
wherein L′ is as defined above, or
R² and R³ form, together with the nitrogen to which they are attached, a 5- to 10-membered heteroaryl or 5- to 10-membered heterocyclyl ring,
wherein:
said phenyl, carbocyclyl, heterocyclyl and heteroaryl groups are optionally fused to a further cyclic moiety selected from phenyl, C₅-C₆ carbocyclyl, 5- to 6-membered heterocyclyl and 5- to 6-membered heteroaryl groups;
the phenyl, heteroaryl, carbocyclyl and heterocyclyl groups and moieties in the groups R¹, R² and that formed by R² and R³ are unsubstituted or substituted by one, two or three substituents which are the same or different and are selected from halogen, hydroxy, amino, thio, C₁-C₆ alkyl, C₂-C₆ alkenyl, nitro, cyano or -Het-L′, wherein Het and L′ are as defined above; and
the alkyl, alkenyl and alkynyl groups and moieties in R¹ to R⁵ are unsubstituted or substituted by one, two or three substituents which are the same or different and are selected from halogen, hydroxy, amino and thio substituents.

2. Use according to claim 1, wherein X represents —CO—, —CO—O—, —CO—S— or —CONR′—.

3. Use according to claim 1 or 2, wherein L represents a bond or a C₁-C₆ alkyl or C₂-C₆ alkenyl moiety.

4. Use according to any one of the preceding claims, wherein L′ represents a C₁-C₆ alkyl or C₂-C₆ alkenyl moiety.

5. Use according to any one of the preceding claims, wherein A represents a phenyl, 5- to 6-membered heteroaryl, C₃-C₆ carbocyclyl or 5- to 6-membered heterocyclyl group, said group being optionally fused to a phenyl, 5- to 6-membered heteroaryl, C₅-C₆ carbocyclyl or 5- to 6-membered heterocyclyl moiety.

6. Use according to any one of the preceding claims, wherein Z is -Het-L′- or —X-L′-.

7. Use according to any one of the preceding claims, wherein R is hydrogen or C₁-C₂ alkyl.

8. Use according to any one of the preceding claims, wherein R¹ represents: (a) -L-A; (b) -L-CR(A)(L-A); (c) -A-A′ or -A-L-A; or (d) -A-Z-A.

9. Use according to any one of the preceding claims, R⁵ represents hydrogen or C₁-C₄ alkyl.

10. Use according to any one of the preceding claims, wherein J is —NMe-, or a direct bond.

11. Use according to any one of the preceding claims, wherein L″ is —X-L′ or —CONH₂.

12. Use according to any one of the preceding claims, wherein R² represents -L-A, -L-A′, -L-A-A′, -L-A-L-A, -L-CR(A)(L-A) or -L-CR(A)(L″).

13. Use according to any one of the preceding claims, wherein R³ represents hydrogen, C₁-C₆ alkyl, C₂-C₆ alkenyl or —(CO)-L′.

14. Use according to any one of the preceding claims, wherein R² and R³ form, together with the nitrogen to which they are attached, a 5- to 6-membered heteroaryl or 5- to 6-membered heterocyclyl ring which can be optionally fused to a phenyl, 5- to 6-membered heteroaryl, C₃-C₅ carbocyclyl or 5- to 6-membered heterocyclyl group.

15. Use according to any one of the preceding claims, wherein:
R¹ represents:
(a) -L-A wherein L is a bond or a C₁-C₂ alkyl moiety and A is a phenyl group;
(b) —CHA₂ wherein A is a phenyl group;
(c) -L-A-A′ wherein L is a bond or a methylene moiety, A′ is —O-A or —C(O)-A and each A is the same or different and is a phenyl or pyridyl group; or
(d) -A-Z-A wherein each A is a phenyl group and Z is —O—C₁-C₂ alkyl-;
J is —NH—, —NMe- or a direct bond;
R⁴ is represents hydrogen or methyl; and either
R² represents -L-A wherein L is a bond or a C₁-C₄ alkyl moiety and A is a phenyl, dihydroindenyl or tetrahydronaphthalenyl group; -L-A′ wherein L is a bond and A′ is —(CO)-phenyl; -L-A-A′ wherein L is a C₁-C₂ alkyl moiety, A′ is —O-A and each A is a phenyl group; -L-A-L-A wherein each L is a bond or a C₁-C₂ alkyl moiety and each A is the same or different and is a phenyl or piperidinyl group; -L-CR(A)(L-phenyl) wherein each L is a bond, A is a phenyl or cyclopropyl group and R is hydrogen; or -L-CR(A)(L″) wherein L is a bond, A is a phenyl group, R is hydrogen and L″ is —X-L′ or —CONH₂, wherein X is —C(O)—O— and L′ is C₁-C₂ alkyl; and
R³ represents hydrogen, C₁-C₂ alkyl or —(CO)-L′, wherein L′ is C₁-C₂ haloalkyl; or
R² and R³ form, together with the nitrogen to which they are attached, a 5- to 10-membered heteroaryl or 5- to 10-membered heterocyclyl ring, they form an tetrahydroisoquinolinyl or isoindoline-1,3-dionyl group,
wherein:
the phenyl, heteroaryl, heterocyclyl and carbocyclyl groups and moieties in the groups R¹, R² and that formed by R² and R³ are unsubstituted or are substituted by one, two or three unsubstituted substituents which are the same or different and are selected from fluorine, chlorine, bromine, C₁-C₄ alkyl, C₂-C₄ alkenyl, C₁-C₂ haloalkyl, —O—(C₁-C₄ alkyl), —O—(C₁-C₄ alkenyl) or —O—(C₁-C₂ haloalkyl) or by a single cyano or hydroxy group; and
the alkyl, alkenyl and alkynyl groups and moieties in R¹ to R⁴ are unsubstituted.

16. Use according to any one of the preceding claims, wherein said condition is chronic or acute pain, a bowel disorder, a bladder dysfunction, tinnitus or a demyelinating disease.

17. A compound of the formula (I) or a pharmaceutically acceptable salt thereof, as defined in any one of claims 1 to 16, for use in a method of treating the human or animal body.

18. A compound of formula (I) as defined in any one of claims 1 to 16 or a pharmaceutically acceptable salt thereof.

19. A pharmaceutical composition comprising a compound of the formula (I), as defined in claim 17, or a pharmaceutically acceptable salt thereof, and a pharmaceutically acceptable carrier or diluent.

20. A composition according to claim 19 which is a capsule or tablet comprising from 10 to 500 mg of a compound of the formula (I), as defined in claim 17, or a pharmaceutically acceptable salt thereof.

21. An inhalation device comprising a pharmaceutical composition according to claim 19.

22. An inhalation device according to claim 21 which is a nebulizer.

23. A method of treating a patient suffering from or susceptible to a condition as defined in claim 1 or 16, which method comprises administering to said patient an effective amount of a compound of formula (I), as defined in any of claims 1 to 15, or a pharmaceutically acceptable salt thereof.
